(19) United States
(12) Patent Application Publication
(10) Pub. No.: US 2024/0398764 A1
(43) Pub. Date: Dec. 5, 2024

(54) METHODS OF TREATING DISEASES ASSOCIATED WITH SENESCENT CELL ACCUMULATION

(71) Applicant: University of Florida Research Foundation, Incorporated, Gainesville (JP)

(72) Inventors: Daohong Zhou, Gainesnille, FL (US); Guangrong Zheng, Gainesville, FL (US); Yonghan He, Gainesville, FL (US); Liya Pi, Gainesville, FL (US)

(21) Appl. No.: 18/700,643

(22) PCT Filed: Oct. 12, 2022
(86) PCT No.: PCT/US2022/046367

§ 371 Date: Apr. 11, 2024

Related U.S. Application Data
(60) Provisional application No. 63/254,978, filed on Oct. 12, 2021.

(51) Int. Cl. A61K 31/427; A61P 1/16; A61P 35/00

(52) U.S. Cl. CPC A61K 31/427; A61P 1/16; A61P 35/00

(57) ABSTRACT

Disclosed herein are methods of treating diseases and disorders associated with senescent cell accumulation (e.g., an age-related disease and/or liver disease, e.g., obesity, liver steatosis, non-alcoholic steatohepatitis (NASH), liver fibrosis, cirrhosis, or hepatocellular carcinoma (HCC)) and methods of ameliorating insulin resistance, pre-diabetes, or diabetes. Also disclosed herein are methods of degrading B-cell lymphoma-2 (Bcl-2) and/or B-cell lymphoma-extra large (Bcl-xl) proteins and methods of killing a senescent cell.

### RELATED APPLICATIONS

The present application claims priority to U.S. Provisional Patent Application No. 63/254,978, filed Oct. 12, 2021, which is incorporated herein by reference.

### GOVERNMENT SUPPORT

This invention was made with government support under Grant Nos. CA219836, CA211963 and AG063801 awarded by the National Institutes of Health. The government has certain rights in the invention.

### BACKGROUND

Senescent cells (SnCs) have recently emerged as therapeutic targets for age-related diseases, including cancer. SnCs accumulate with age and as a result of exposure to a variety of stressors, including radiation and chemotherapy. They play a causal role in many age-related diseases (such as neurodegenerative and cardiovascular diseases, osteoarthritis, and cancer) via expression of the senescence-associated secretory phenotype (SASP) to secrete various inflammatory mediators and proteases. Small molecules that can selectively kill SnCs, termed senolytics, have the potential to prevent and treat a growing number of age-related diseases and extend lifespan. To date, several classes of senolytics have been identified, including synthetic small-molecule therapeutics, such as inhibitors of the anti-apoptotic B-cell lymphoma-2 (Bcl-2) family of proteins. Proteins of the Bcl-2 family, including B-cell lymphoma-extra large (Bcl-xl) protein, regulate apoptosis and have been implicated in various cancers, as well as resistance to cancer treatments.

Most synthetic small-molecule senolytics are repurposed anticancer drugs that have various on-target and/or off-target toxicities in vivo, limiting their clinical use as anti-aging agents. Therefore, strategies to reduce on-target and/or off-target toxicity of known senolytics are urgently needed.

### SUMMARY

It is now discovered that compounds described herein possess heretofore unknown functional activity relevant for addressing disease and disorders of the liver. The present disclosure relates to methods of treating, ameliorating, and/or preventing a disease associated with senescent cell accumulation (e.g., an age-related disease and/or liver disease). The present disclosure also provides methods of degrading B-cell lymphoma-2 (Bcl-2) and B-cell lymphoma-extra large (Bcl-xl) proteins, and methods of killing senescent cells.

In one aspect, provided herein is a method of treating and/or preventing a disease or disorder in a subject identified in need thereof, the method comprising administering an effective amount of a compound of the formula:

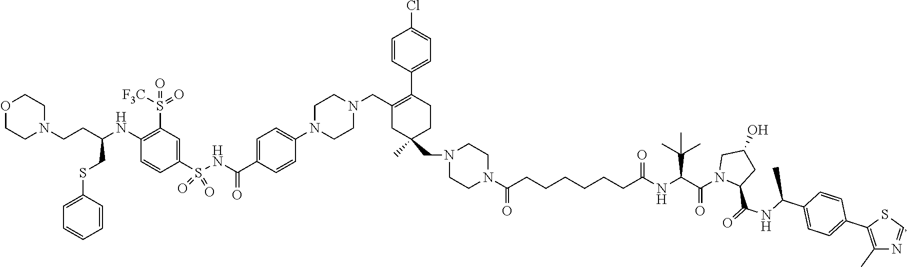 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt thereof, or a pharmaceutical composition thereof.

In another aspect, provided herein is a method of treating a subject suffering from or susceptible to a disease or disorder, the method comprising administering an effective amount of a compound of the formula:

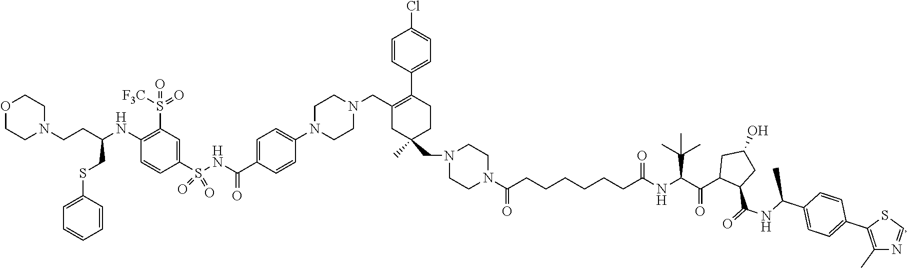 [SMILES: Cc1ncsc1-c1ccc([C@H](C)NC(=O)[C@@H]2C[C@@H](O)CC2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt thereof, or a pharmaceutical composition thereof.

In certain embodiments, the disease is a disease associated with senescent cell accumulation. In certain embodiments, the disease is an age-related disease. In certain embodiments, the disease is a liver disease. In some embodiments, the disease is an alcohol-induced liver disease or a non-alcoholic fatty liver disease. In certain embodiments, the disease is a diabetes- and/or obesity-related non-alcoholic fatty liver disease. In some embodiments, the disease is liver steatosis, non-alcoholic steatohepatitis (NASH), liver fibrosis, cirrhosis, or liver cancer

In another aspect, provided herein is a method of ameliorating insulin resistance in a subject identified as in need thereof, comprising administering an effective amount of a compound of the formula:

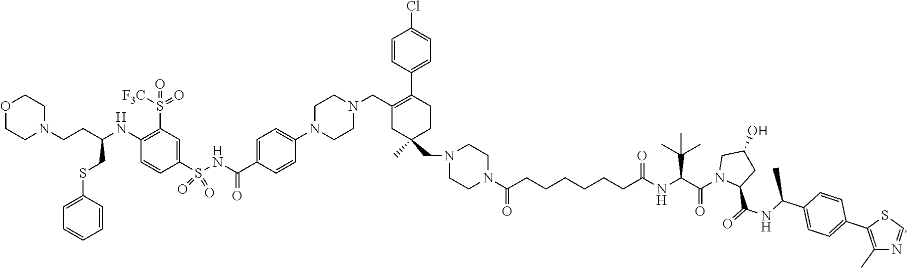 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt thereof, or a pharmaceutical composition thereof.

In another aspect, provided herein is a method of ameliorating pre-diabetes, diabetes, and/or obesity in a subject identified as in need thereof, comprising administering an effective amount of a compound of the formula:

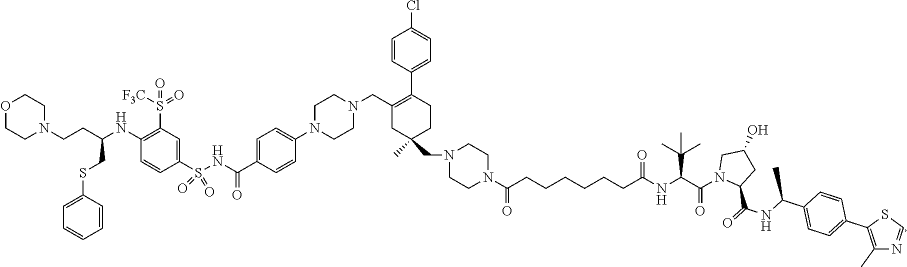 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt thereof, or a pharmaceutical composition thereof.

In another aspect, provided herein is a method of degrading B-cell lymphoma-2 (Bel-2) and/or B-cell lymphoma-extra large (Bel-xl) proteins, the method comprising contacting Bel-2 and/or Bcl-xl with an effective amount of a compound of the formula:

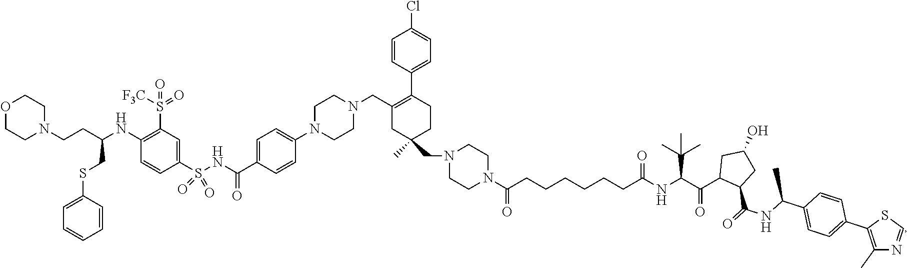 [SMILES: Cc1ncsc1-c1ccc([C@H](C)NC(=O)[C@@H]2C[C@@H](O)CC2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt thereof, or a pharmaceutical composition thereof.

In another aspect, provided herein is a method of killing a senescent cell, the method comprising contacting the senescent cell with an effective amount of a compound of the formula:

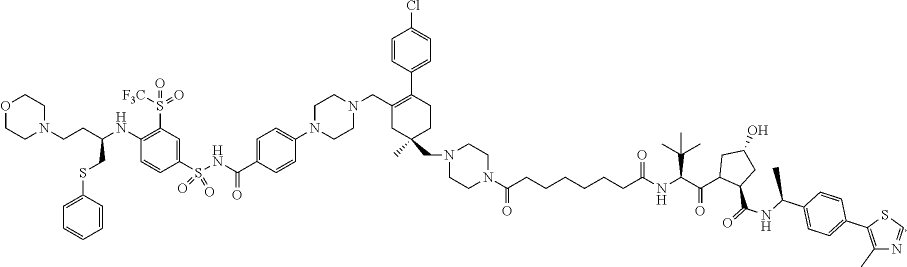 [SMILES: Cc1ncsc1-c1ccc([C@H](C)NC(=O)[C@@H]2C[C@@H](O)CC2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt thereof, or a pharmaceutical composition thereof.

The details of certain embodiments of the invention are set forth in the Detailed Description of Certain Embodiments, as described below. Other features, objects, and advantages of the invention will be apparent from the Definitions, Examples, Figures, and Claims. It should be understood that the aspects described herein are not limited to specific embodiments, methods, or configurations, and as such can, of course, vary. It is also to be understood that the terminology used herein is for the purpose of describing particular aspects only and, unless specifically defined herein, is not intended to be limiting.

### BRIEF DESCRIPTION OF THE DRAWINGS

The accompanying drawings, which constitute a part of this specification, illustrate several embodiments of the invention and together with the description, provide non-limiting examples of the invention.

FIG. 1A depicts immunoblots showing degradation of Bcl-xl and Bcl-2 in HEK293T cells treated with various concentrations of 753b for 16 hours. FIG. 1B depicts immunoblots showing degradation of Bcl-xl and Bcl-2 in HEK293T cells treated with 1 μM 753b for various durations. 3-actin was used as a loading control.

FIG. 2A depicts immunoblots showing degradation of Bcl-xl and Bcl-2 in non-senescent WI-38 fibroblast cells (Non-SnCs) treated with varying concentrations of 753b. 3-actin was used as a loading control. FIG. 2B depicts viability of Non-SnCs treated with varying concentrations of 753b or ABT263. FIG. 2C depicts immunoblots showing degradation of Bcl-xl and Bcl-2 in ionizing radiation-induced senescent WI-38 fibroblast cells (IR-SnCs) treated with varying concentrations of 753b. 3-actin was used as a loading control. FIG. 2D depicts viability of IR-SnCs treated with varying concentrations of 753b or ABT263. FIG. 2E depicts immunoblots showing degradation of Bcl-xl and Bcl-2 in human platelets treated with varying concentrations of 753b. FIG. 2F depicts viability of human platelets treated with varying concentrations of 753b or ABT263.

FIGS. 3A to 3B depicts viability of non-SnC human umbilical vein endothelial cells (HUVEC) and IR-induced SnC HUVEC treated with varying concentrations of ABT263 (FIG. 3A) or 753b (FIG. 3B). FIGS. 3C to 3D depict viability of non-SnC renal epithelial cells (REC), IR-induced SnC REC, and replicative senescent REC treated with varying concentrations of ABT263 (FIG. 3C) or 753b (FIG. 3D).

FIGS. 4A to 4E depict expression of Cdkn2a (p16) mRNA in the liver (FIG. 4A), spleen (FIG. 4B), lung (FIG. 4C), kidney (FIG. 4D), and fat (FIG. 4E) tissues of naturally aged mice treated with vehicle or 5 mg/kg 753b. ****, p<0.0001

FIG. 5 depicts the experimental design of C57BL6 mice with induced diabetes and fed high fat diet (HFD) and treated with either 753b or vehicle.

FIG. 6A shows a Western blot of Bcl-xl levels in the livers of STZ/HFD mice treated with 753b or vehicle control. GAPDH was used as a loading control. FIG. 6B depicts the relative expression of p16 and p21 at mRNA levels in the livers of STZ/HFD mice treated with 753b or vehicle control. *p<0.05. FIGS. 6C to 6D show senescence-associated beta-galactosidase (SA-β-gal) stained livers of STZ/HFD mice treated with vehicle control (FIG. 6C) or 753b (FIG. 6D). Scale bar: 150 mm.

FIG. 7A shows images of liver tissues of STZ/HFD mice treated with vehicle control or 753b. Scale bar: 150 mm. FIG. 7B depicts percentage of oil red⁺ area/field of the images depicted in FIG. 7A. Values represented means±SD. *p<0.05. FIG. 7C shows hepatic triglyceride levels (mg/g) in STZ/HFD mice treated with vehicle control or 753b. Values represented means±SD. *p<0.05.

FIG. 8A is an image showing body condition of STZ/HFD mice treated with vehicle control or 753b. FIG. 8B depicts blood glucose (mg/dL) levels versus time after intraperitoneal injection of insulin in STZ/HFD mice treated with vehicle control or 753b. Data were means±SD from 5 mice per group. *p<0.05.

FIG. 9A depicts relative change in aSMA, Procollagen-1, Tgf-β, Ctgf, and Pdgf-β genes in livers of STZ/HFD mice treated with vehicle control or 753b. Data were means±SD of triplicate experiments. *p<0.05. FIG. 9B shows images of immunofluorescent staining of aSMA (top) or Sirius red staining for collagen fibril (bottom). Scale bar: 200 mm. FIG. 9C shows a Western blot of aSMA and pro-collagen type I proteins in livers of STZ/HFD mice treated with 753b. Actin was used as a loading control. FIG. 9D depicts hydroxyproline (μg/g) levels in the liver of STZ/HFD mice treated with vehicle control or 753b. *p<0.05.

FIG. 10A depicts images of livers of STZ/HFD mice treated with vehicle control or 753b (top) and MRIs of T1 pre-contrast and T2-weighted images of corresponding livers (bottom). Tumor lesions are indicated by yellow arrows. FIG. 10B shows tumor number and volume (cm³) in livers of STZ/HFD mice treated with vehicle control or 753b. *p<0.05. FIG. 10C depicts a Western blot of Gpc3 in livers of STZ/HFD mice treated with vehicle control or 753b. Actin was used as a loading control. FIG. 10D shows the ratio of liver weight to body weight in STZ/HFD mice treated with vehicle control or 753b.

### DEFINITIONS

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and ·x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds herein may also contain linkages (e.g., carbon-carbon bonds) wherein bond rotation is restricted about that particular linkage, e.g. restriction resulting from the presence of a ring or double bond. Accordingly, all cis/trans and E/Z isomers are expressly included in the present disclosure. The compounds herein may also be represented in multiple tautomeric forms, in such instances, the invention expressly includes all tautomeric forms of the compounds described herein, even though only a single tautomeric form may be represented. While compounds may be depicted as racemic or as one or more diastereoisomers, enantiomers, or other isomers, all such racemic, diastereoisomer, enantiomer, or other isomer forms of that depicted are included in the present disclosure. All such isomeric forms of such compounds herein are expressly included in the present disclosure. All crystal forms and polymorphs of the compounds described herein are expressly included in the present invention. The term “isomers” is intended to include diastereoisomers, enantiomers, regioisomers, structural isomers, rotational isomers, tautomers, and the like. For compounds which contain one or more stereogenic centers, e.g., chiral compounds, the methods of the invention may be carried out with an enantiomerically enriched compound, a racemate, or a mixture of diastereomers.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “co-crystal” refers to a crystalline structure comprising at least two different components (e.g., a compound and an acid), wherein each of the components is independently an atom, ion, or molecule. In certain embodiments, none of the components is a solvent. In certain embodiments, at least one of the components is a solvent. A co-crystal of a compound and an acid is different from a salt formed from a compound and the acid. In the salt, a compound is complexed with the acid in a way that proton transfer (e.g., a complete proton transfer) from the acid to a compound easily occurs at room temperature. In the co-crystal, however, a compound is complexed with the acid in a way that proton transfer from the acid to a herein does not easily occur at room temperature. In certain embodiments, in the co-crystal, there is substantially no proton transfer from the acid to a compound. In certain embodiments, in the co-crystal, there is partial proton transfer from the acid to a compound. Co-crystals may be useful to improve the properties (e.g., solubility, stability, and ease of formulation) of a compound.

The term “isotopically labeled compound” refers to a derivative of a compound that only structurally differs from the compound in that at least one atom of the derivative includes at least one isotope enriched above (e.g., enriched between 3- and 10-fold, between 10- and 30-fold, between 30- and 100-fold, between 100- and 300-fold, between 300- and 1,000-fold, between 1,000- and 3,000-fold, or between 3,000- and 10,000-fold above) its natural abundance, whereas each atom of the compound includes isotopes at their natural abundances. In certain embodiments, the isotope enriched above its natural abundance is ²H. In certain embodiments, only one, two, three, four, or five hydrogen atoms of the isotopically labeled compound include ²H above its natural abundance. In certain embodiments, the isotope enriched above its natural abundance is ¹³C, ¹⁵N, ¹⁸O, or ¹⁸F. In certain embodiments, only one, two, or three carbon atoms of the isotopically labeled compound include ¹³C above its natural abundance. In certain embodiments, only one, two, or three nitrogen atoms of the isotopically labeled compound include ¹⁵N above its natural abundance. In certain embodiments, only one, two, or three oxygen atoms of the isotopically labeled compound include ¹⁸O above its natural abundance. In certain embodiments, only one, two, or three fluorine atoms of the isotopically labeled compound include ¹⁸F above its natural abundance.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgaard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds described herein may be preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “target tissue” refers to any biological tissue of a subject (including a group of cells, a body part, or an organ) or a part thereof, including blood and/or lymph vessels, which is the object to which a compound, particle, and/or composition of the invention is delivered. A target tissue may be an abnormal or unhealthy tissue, which may need to be treated. A target tissue may also be a normal or healthy tissue that is under a higher than normal risk of becoming abnormal or unhealthy, which may need to be prevented. In certain embodiments, the target tissue is the liver. In certain embodiments, the target tissue is the lung. A “non-target tissue” is any biological tissue of a subject (including a group of cells, a body part, or an organ) or a part thereof, including blood and/or lymph vessels, which is not a target tissue.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence. In some embodiments, “treating” includes amelioration, cure, preventing the disease or disorder from becoming worse, or slowing the rate of progression.

The term “ameliorate,” as used herein, is intended to mean improve, lessen, inhibit, delay onset of and/or otherwise modulate a disease or disorder.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

An “active ingredient” as described herein is a compound that is biologically active or pharmacologically active or has other direct effect in the diagnosis, cure, mitigation, amelioration, treatment, or prevention of disease, or to affect the structure or any function of a subject, and all biological components therein.

As used herein, “inhibiting” encompasses reducing and halting progression.

The term “modulate” refers to increases or decreases in the activity of a cell in response to exposure to a compound disclosed herein

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high-performance liquid chromatography. Particularly, in embodiments the compound is at least 85% pure, more preferably at least 90% pure, more preferably at least 95% pure, more preferably at least 98% pure, and most preferably at least 99% pure.

The terms “polypeptide,” “peptide” and “protein” are used interchangeably herein to refer to a polymer of amino acid residues. The terms apply to amino acid polymers in which one or more amino acid residue is an artificial 5 chemical mimetic of a corresponding naturally occurring amino acid, as well as to naturally occurring amino acid polymers and non-naturally occurring amino acid polymer.

The term “protein” refers to series of amino acid residues connected one to the other by peptide bonds between the alpha-amino and carboxy groups of adjacent 10 residues.

The term “proteolysis-targeting chimera” or “PROTAC” refers to a heterobifunctional molecule capable of inducing intracellular proteolysis. In some embodiments, a PROTAC comprises an E3-ubiquitin ligase binding molecule covalently linked to a component that binds the protein targeted for degradation.

The term “liver disease” or “hepatic disease” refers to damage to or a disease of the liver. Non-limiting examples of liver disease include intrahepatic cholestasis (e.g., alagille syndrome, biliary liver cirrhosis), fatty liver (e.g., alcoholic fatty liver, Reye's syndrome), hepatic vein thrombosis, hepatolenticular degeneration (i.e., Wilson's disease), hepatomegaly, liver abscess (e.g., amebic liver abscess), liver cirrhosis (e.g., alcoholic, biliary, and experimental liver cirrhosis), alcoholic liver diseases (e.g., fatty liver, hepatitis, cirrhosis), parasitic liver disease (e.g., hepatic echinococcosis, fascioliasis, amebic liver abscess), jaundice (e.g., hemolytic, hepatocellular, cholestatic jaundice), cholestasis, portal hypertension, liver enlargement, ascites, hepatitis (e.g., alcoholic hepatitis, animal hepatitis, chronic hepatitis (e.g., autoimmune, hepatitis B, hepatitis C, hepatitis D, drug induced chronic hepatitis), toxic hepatitis, viral human hepatitis (e.g., hepatitis A, hepatitis B, hepatitis C, hepatitis D, hepatitis E), granulomatous hepatitis, secondary biliary cirrhosis, hepatic encephalopathy, varices, primary biliary cirrhosis, primary sclerosing cholangitis, hepatocellular adenoma, hemangiomas, bile stones, liver failure (e.g., hepatic encephalopathy, acute liver failure), angiomyolipoma, calcified liver metastases, cystic liver metastases, fibrolamellar hepatocarcinoma, hepatic adenoma, hepatoma, hepatic cysts (e.g., Simple cysts, Polycystic liver disease, hepatobiliary cystadenoma, choledochal cyst), mesenchymal tumors (mesenchymal hamartoma, infantile hemangioendothelioma, hemangioma, peliosis hepatis, lipomas, inflammatory pseudotumor), epithelial tumors (e.g., bile duct hamartoma, bile duct adenoma), focal nodular hyperplasia, nodular regenerative hyperplasia, hepatoblastoma, hepatocellular carcinoma, cholangiocarcinoma, cystadenocarcinoma, tumors of blood vessels, angiosarcoma, Karposi's sarcoma, hemangioendothelioma, embryonal sarcoma, fibrosarcoma, leiomyosarcoma, rhabdomyosarcoma, carcinosarcoma, teratoma, carcinoid, squamous carcinoma, primary lymphoma, peliosis hepatis, erythrohepatic porphyria, hepatic porphyria (e.g., acute intermittent porphyria, porphyria cutanea tarda), and Zellweger syndrome.

As used herein, the term “non-alcoholic fatty liver disease” refers to fatty liver cases in which there is no history of alcohol consumption or in which alcohol consumption is not related to the occurrence. The fatty liver refers to a phenomenon in which there is abnormal accumulation of triglyceride in liver cells, compared to normal levels of triglyceride. About 5% of normal liver consists of fat tissue and the main components of the fat are triglycerides, fatty acids, phospholipids, cholesterols, and cholesterol esters. However, once the fatty liver occurs, most of the components are replaced with triglyceride. If the amount of triglycerides is more than 5% of the liver weight, it is diagnosed as fatty liver. The fatty liver is caused by a lipid metabolism disorder or a defect in the process of carrying excessive fat in the liver cells, and is mainly caused by disorders of lipid metabolism in the liver. Most of the fat accumulated in the fatty liver may be a triglyceride. The non-alcoholic fatty liver disease includes non-alcoholic fatty liver, nonalcoholic steatohepatitis, cirrhosis, liver cancer, and the like, but the fatty liver disease to be prevented or treated with the composition of the present invention is included without limitation. In some embodiments, non-alcoholic fatty liver disease refers to a condition characterized by fatty inflammation of the liver that is not due to excessive alcohol use (for example, alcohol consumption of over 20 g/day). In certain embodiments, provided herein nonalcoholic fatty liver disease is related to insulin resistance and the metabolic syndrome.

The term “alcohol-induced liver disease” refers to a disease caused by damage to liver tissue due to excessive ingestion of alcohol, resulting in decreased liver function. In some embodiments, alcohol-induced liver disease refers to complications related to cirrhosis and cirrhosis due to acute liver damage caused by drinking and irreversible change. The initial findings of alcohol-induced liver damage are fatty liver, alcoholic hepatitis caused by repetitive drinking, and it is known to progress to cirrhosis (Hwang, Sung-Kyu, Korean Society of Liver Science, Single Topic Symposium, 2003). Therefore, examples of the alcohol-induced liver disease include, but are not limited to, alcoholic fatty liver, alcoholic hepatitis, liver fibrosis, cirrhosis of the liver, and the like.

The term “spleen disease” refers to a disease of the spleen. Example of spleen diseases include, but are not limited to, splenomegaly, spleen cancer, asplenia, spleen trauma, idiopathic purpura, Felty's syndrome, Hodgkin's disease, and immune-mediated destruction of the spleen.

A “diabetic condition” refers to diabetes and pre-diabetes. Diabetes refers to a group of metabolic diseases in which a person has high blood sugar, either because the body does not produce enough insulin, or because cells do not respond to the insulin that is produced. This high blood sugar produces the classical symptoms of polyuria (frequent urination), polydipsia (increased thirst) and polyphagia (increased hunger). There are several types of diabetes. Type I diabetes results from the body's failure to produce insulin, and presently requires the person to inject insulin or wear an insulin pump. Type II diabetes results from insulin resistance a condition in which cells fail to use insulin properly, sometimes combined with an absolute insulin deficiency. Gestational diabetes occurs when pregnant women without a previous diagnosis of diabetes develop a high blood glucose level. Other forms of diabetes include congenital diabetes, which is due to genetic defects of insulin secretion, cystic fibrosis-related diabetes, steroid diabetes induced by high doses of glucocorticoids, and several forms of monogenic diabetes, e.g., mature onset diabetes of the young (e.g., MODY 1, 2, 3, 4, 5, 6, 7, 8, 9, or 10). Pre-diabetes indicates a condition that occurs when a person's blood glucose levels are higher than normal but not high enough for a diagnosis of diabetes. All forms of diabetes increase the risk of long-term complications. These typically develop after many years, but may be the first symptom in those who have otherwise not received a diagnosis before that time. The major long-term complications relate to damage to blood vessels. Diabetes doubles the risk of cardiovascular disease and macrovascular diseases such as ischemic heart disease (angina, myocardial infarction), stroke, and peripheral vascular disease. Diabetes also causes microvascular complications, e.g., damage to the small blood vessels. Diabetic retinopathy, which affects blood vessel formation in the retina of the eye, can lead to visual symptoms, reduced vision, and potentially blindness. Diabetic nephropathy, the impact of diabetes on the kidneys, can lead to scarring changes in the kidney tissue, loss of small or progressively larger amounts of protein in the urine, and eventually chronic kidney disease requiring dialysis. Diabetic neuropathy is the impact of diabetes on the nervous system, most commonly causing numbness, tingling and pain in the feet and also increasing the risk of skin damage due to altered sensation. Together with vascular disease in the legs, neuropathy contributes to the risk of diabetes-related foot problems, e.g., diabetic foot ulcers, that can be difficult to treat and occasionally require amputation.

Unless otherwise required by context, singular terms shall include pluralities, and plural terms shall include the singular. The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

When a range of values (“range”) is listed, it encompasses each value and sub-range within the range. A range is inclusive of the values at the two ends of the range unless otherwise provided. It will be understood that when a range is recited in the application, the ends of the range are specifically disclosed as if specifically recited. For example, a range of about 19% to about 99% specifically include a disclosure separately of 19% and separately of 99%.

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2%, or 1% of a given value or range of values.

### DETAILED DESCRIPTION OF CERTAIN EMBODIMENTS

Before the disclosed systems, compounds, combinations, compositions, methods, uses, and kits are described in more detail, it should be understood that the aspects described herein are not limited to specific embodiments, methods, apparati, or configurations, and as such can, of course, vary. It is also to be understood that the terminology used herein is for the purpose of describing particular aspects only and, unless specifically defined herein, is not intended to be limiting.

The compounds, compositions, and kits used in the methods provided herein can provide for the treatment, prevention, or amelioration of a disease or disorder (e.g., a disease associated with senescent cell accumulation (e.g., age-related disease and/or liver disease)). Additionally, the compounds, compositions, and kits used in the methods provided herein can ameliorate insulin resistance, pre-diabetes, diabetes, and/or obesity. The compounds, compositions, methods, and kits may degrade B-cell lymphoma-2 (Bcl-2) and/or B-cell lymphoma-extra large (Bcl-xl) proteins. The compounds, compositions, methods, and kits may kill senescent cells, which are implicated in many age-related diseases (such as neurodegenerative and cardiovascular diseases, osteoarthritis, and cancer).

### Methods of Use

In one aspect, the present disclosure provides a method of treating a disease or disorder in a subject identified in need thereof, the method comprising administering an effective amount of a compound of the formula:

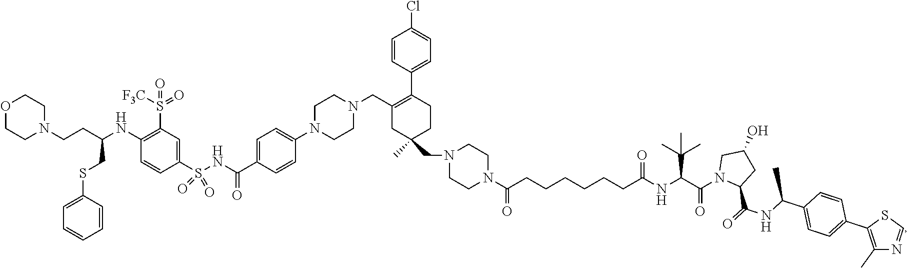 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt, solvate, hydrate, polymorph, co-crystal, tautomer, stereoisomer, isotopically labeled compound, or prodrug thereof, or a pharmaceutical composition thereof.

In another aspect, the present disclosure provides a method of preventing a disease or disorder in a subject identified in need thereof, the method comprising administering an effective amount of a compound of the formula:

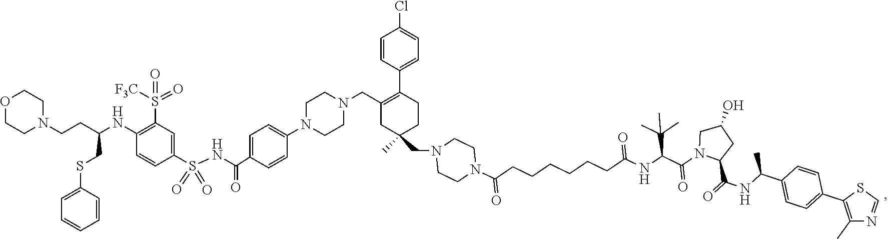 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt, solvate, hydrate, polymorph, co-crystal, tautomer, stereoisomer, isotopically labeled compound, or prodrug thereof, or a pharmaceutical composition thereof.

In certain embodiments, the method comprises administering an effective amount of the compound, or a pharmaceutically acceptable salt thereof, or a pharmaceutical composition thereof.

Provided herein are methods of treating and/or preventing a disease or disorder in a subject identified in need thereof, the method comprising administering an effective amount of a compound of the formula:

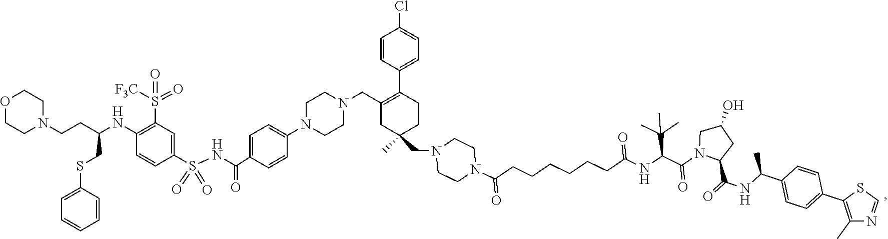 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt thereof, or a pharmaceutical composition thereof.

Also provided herein are methods of treating a subject suffering from or susceptible to a disease or disorder, the method comprising administering an effective amount of a compound of the formula:

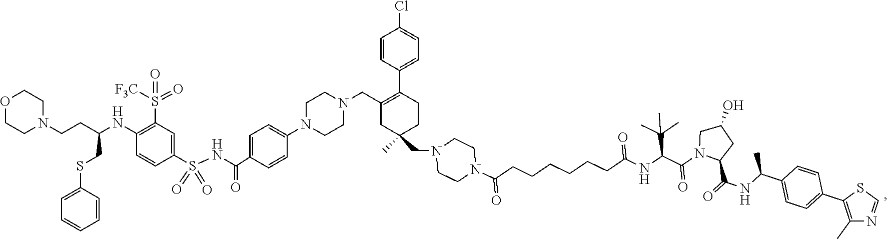 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt thereof, or a pharmaceutical composition thereof.

In another aspect, the present disclosure provides a method of ameliorating insulin resistance in a subject identified as in need thereof, comprising administering an effective amount of a compound of the formula:

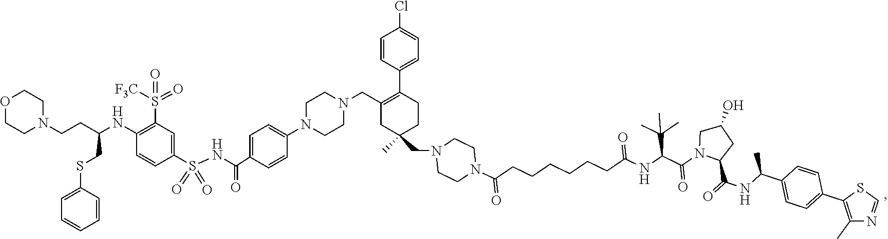 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt, solvate, hydrate, polymorph, co-crystal, tautomer, stereoisomer, isotopically labeled compound, or prodrug thereof, or a pharmaceutical composition thereof.

Also provided herein are methods of ameliorating insulin resistance in a subject identified as in need thereof, comprising administering an effective amount of a compound of the formula:

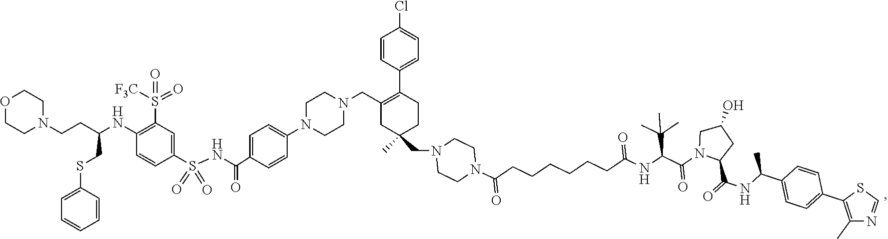 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt thereof, or a pharmaceutical composition thereof. In certain embodiments, the blood glucose level of a subject in need is at least 500 mg/dL, at least 400 mg/dL, at least 300 mg/dL, at least 200 mg/dL, or at least 100 mg/dL prior to administration. In certain embodiments, the blood glucose level of a subject in need is about 5 mg/dL to about 100 mg/dL, about 5 mg/dL to about 200 mg/dL, about 5 mg/dL to about 300 mg/dL, about 5 mg/mL to about 400 mg/dL, about 5 mg/dL to about 500 mg/dL, or about 5 mg/mL to about 1000 mg/dL prior to administration.

In certain embodiments, insulin resistance is determined via an injected insulin tolerance test, wherein insulin is administered concurrently with or after administration of the compound. In certain embodiments, the blood glucose level of a subject is less than 200 mg/dL, less than 190 mg/dL, less than 180 mg/dL, less than 170 mg/dL, less than 160 mg/dL, less than 150 mg/dL, less than 140 mg/dL, less than 130 mg/dL, less than 120 mg/dL, less than 90 mg/dL, less than 80 mg/dL, less than 70 mg/dL, less than 60 mg/dL, or less than 50 mg/dL 90 minutes after administration of insulin. In certain embodiments, the blood glucose level is about 5 mg/dL to about 100 mg/dL, about 5 mg/dL to about 90 mg/dL, about 5 mg/dL to about 80 mg/dL, about 5 mg/dL to about 70 mg/dL, about 5 mg/dL to about 60 mg/dL, or about 5 mg/d to about 50 mg/dL 90 minutes after administration of insulin. In certain embodiments, blood glucose level of a subject is less than 200 mg/dL, less than 190 mg/dL, less than 180 mg/dL, less than 170 mg/dL, less than 160 mg/dL, less than 150 mg/dL, less than 140 mg/dL, less than 130 mg/dL, less than 120 mg/dL, less than 90 mg/dL, less than 80 mg/dL, less than 70 mg/dL, less than 60 mg/dL, or less than 50 mg/dL 60 minutes after administration of insulin. In certain embodiments, the blood glucose level is about 5 mg/dL to about 100 mg/dL, about 5 mg/dL to about 90 mg/dL, about 5 mg/dL to about 80 mg/dL, about 5 mg/dL to about 70 mg/dL, about 5 mg/dL to about 60 mg/dL, or about 5 mg/dL to about 50 mg/dL 60 minutes after administration of insulin. In certain embodiments, blood glucose level of a subject is less than 200 mg/dL, less than 190 mg/dL, less than 180 mg/dL, less than 170 mg/dL, less than 160 mg/dL, less than 150 mg/dL, less than 140 mg/dL, less than 130 mg/dL, less than 120 mg/dL, less than 90 mg/dL, less than 80 mg/dL, less than 70 mg/dL, less than 60 mg/dL, or less than 50 mg/dL 30 minutes after administration of insulin. In certain embodiments, the blood glucose level is about 5 mg/dL to about 100 mg/dL, about 5 mg/dL to about 90 mg/dL, about 5 mg/dL to about 80 mg/dL, about 5 mg/dL to about 70 mg/dL, about 5 mg/dL to about 60 mg/dL, or about 5 mg/dL to about 50 mg/dL 30 minutes after administration of insulin.

In another aspect, the present disclosure provides a method of ameliorating pre-diabetes, diabetes, and/or obesity in a subject identified as in need thereof, comprising administering an effective amount of a compound of the formula:

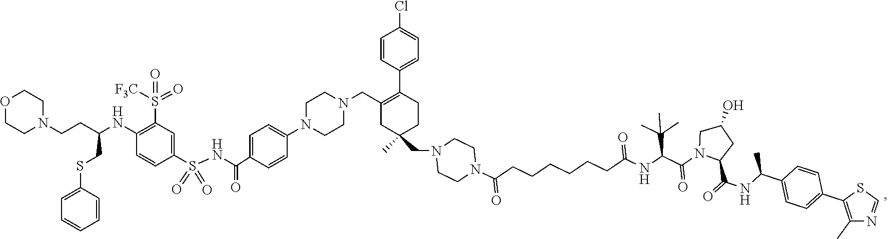 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt, solvate, hydrate, polymorph, co-crystal, tautomer, stereoisomer, isotopically labeled compound, or prodrug thereof, or a pharmaceutical composition thereof.

Also provided herein are methods of ameliorating pre-diabetes, diabetes, and/or obesity in a subject identified as in need thereof, comprising administering an effective amount of a compound of the formula:

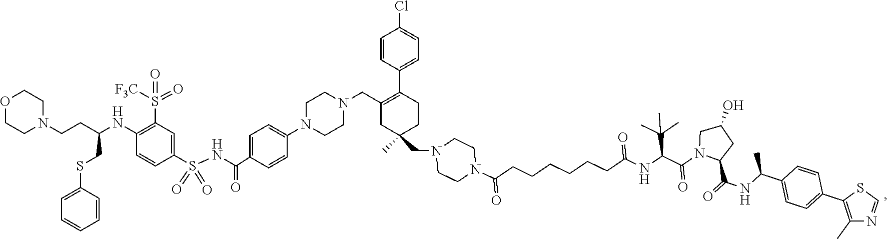 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt thereof, or a pharmaceutical composition thereof.

In some embodiments, the method further comprises suppressing hepatic fat accumulation. In certain embodiments, hepatic fat accumulation is determined based on the level of hepatic triglyceride in liver tissue. In some embodiments, hepatic fat accumulation is determined by quantifying stained liver images. In certain embodiments, the concentration of hepatic triglyceride in a treated tissue is less than 60 mg/g, less than 55 mg/g, less than 50 mg/g, less than 45 mg/g, less than 40 mg/g, less than 35 mg/g, less than 30 mg/g, less than 25 mg/g, or less than 20 mg/g. In certain embodiments, the concentration of hepatic triglyceride in a treated tissue is less than 60 mg/g, less than 55 mg/g, less than 50 mg/g, less than 45 mg/g, less than 40 mg/g, or less than 35 mg/g.

In some embodiments, the method further comprises preventing insulin resistance. In certain embodiments, insulin resistance is determined via an injected insulin tolerance test.

In some embodiments, the disease is associated with senescent cell accumulation. In certain embodiments, the disease is an age-related disease and/or a liver disease. In some embodiments, the disease is an age-related disease. In certain embodiments, the disease is a liver disease. In some embodiments, the disease is an alcohol-induced liver disease or a non-alcoholic fatty liver disease. In certain embodiments, the disease is an alcohol-induced liver disease. In some embodiments, the disease is a non-alcoholic fatty liver disease. In certain embodiments, the non-alcoholic fatty liver disease is a diabetes- and/or obesity-related non-alcoholic fatty liver disease. In some embodiments, the disease is liver steatosis, non-alcoholic steatohepatitis (NASH), liver fibrosis, cirrhosis, or liver cancer.

In certain embodiments, the disease is liver steatosis, NASH, liver fibrosis, or cirrhosis. In certain embodiments, the disease is NASH, liver fibrosis, or cirrhosis. In some embodiments, the disease is NASH. In certain embodiments, the disease is liver fibrosis. In some embodiments, the compound inhibits myofibroblast cell activation. In some embodiments, administration of the compound reduces the level of fibrosis-related genes in the liver. In some embodiments, administration of the compound reduces the level of one or more of aSMA, procollagen-I, Tgf-β, Ctaf, or Pdgf-b. In some embodiments, administration of the compound produces lower level of fibrosis-related genes in the liver by at least 10%, at least 20%, at least 30%, at least 50%, at least 60%, at least 70%, at least 80%, or at least 90%. In some embodiments, administration of the compound reduces the level of one or more of aSMA, procollagen-I, Tgf-β, Ctaf, or Pdgf-b by at least 10%, at least 20%, at least 30%, at least 50%, at least 60%, at least 70%, at least 80%, or at least 90%.

In some embodiments, the disease is cirrhosis. In some embodiments, the disease is liver cancer. In certain embodiments, the disease is hepatocellular carcinoma (HCC). In some embodiments, the compound reduces tumorigenesis. In some embodiments, the compound reduces the number of tumors. In some embodiments, the compound reduces tumor volume by at least 10%, about 20%, about 30%, about 40%, about 50%, or about 60%.

In some embodiments, the disease is not cancer. In some embodiments, the disease is not a solid tumor cancer.

In some embodiments, the method comprises ameliorating pre-diabetes and/or diabetes. In some embodiments, the method comprises ameliorating pre-diabetes and/or obesity. In certain embodiments, the method comprises ameliorating diabetes and/or obesity. In some embodiments, the method comprises ameliorating pre-diabetes. In certain embodiments, the method comprises ameliorating diabetes. In some embodiments, the method comprises ameliorating obesity.

In another aspect, the present disclosure provides a method of degrading B-cell lymphoma-2 (Bcl-2) and/or B-cell lymphoma-extra large (Bcl-xl) proteins, the method comprising contacting Bcl-2 and/or Bcl-xl with an effective amount of a compound of the formula:

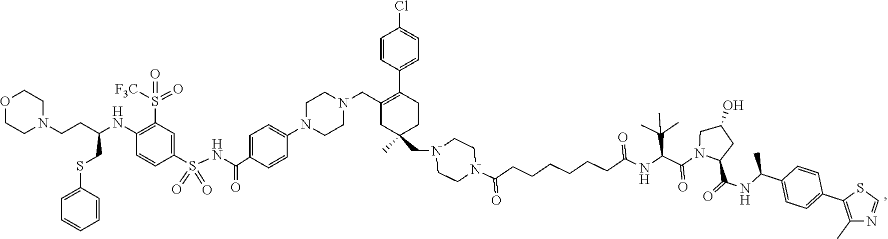 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt, solvate, hydrate, polymorph, co-crystal, tautomer, stereoisomer, isotopically labeled compound, or prodrug thereof, or a pharmaceutical composition thereof.

In certain embodiments, the method comprises contacting Bcl-2 and/or Bcl-xl with an effective amount of the compound, or a pharmaceutically acceptable salt thereof, or a pharmaceutical composition thereof.

Also provided herein are methods of degrading B-cell lymphoma-2 (Bcl-2) and/or B-cell lymphoma-extra large (Bcl-xl) proteins, the method comprising contacting BCL-2 and/or Bcl-xl with an effective amount of a compound of the formula:

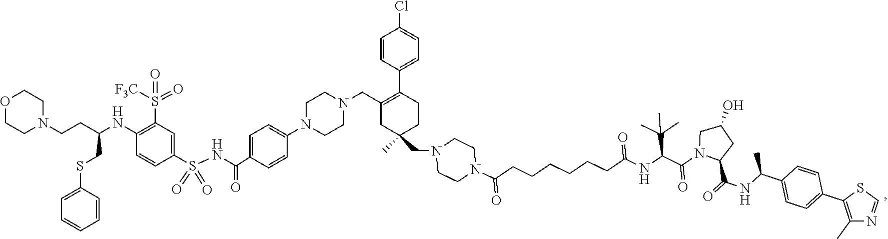 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt thereof, or a pharmaceutical composition thereof. In some embodiments, the protein is Bel-xl. In certain embodiments, the protein is Bcl-2.

In certain embodiments, the compound degrades up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% of Bcl-2 at a compound concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less.

In certain embodiments, the compound degrades up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% of Bcl-xl at a compound concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less.

In certain embodiments, the method further comprises administering the compound or a pharmaceutically acceptable salt, solvate, hydrate, polymorph, co-crystal, tautomer, stereoisomer, isotopically labeled compound, or prodrug thereof, or a pharmaceutical composition thereof, to a subject.

In another aspect, the present disclosure provides a method of killing a senescent cell, the method comprising contacting the senescent cell with an effective amount of a compound of the formula:

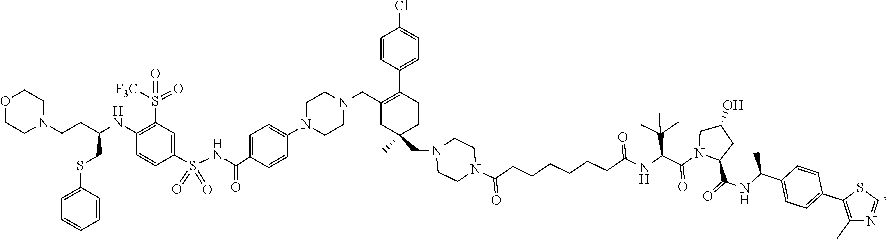 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt, solvate, hydrate, polymorph, co-crystal, tautomer, stereoisomer, isotopically labeled compound, or prodrug thereof, or a pharmaceutical composition thereof.

In certain embodiments, the method comprises contacting the senescent cell with an effective amount of the compound, or a pharmaceutically acceptable salt thereof, or a pharmaceutical composition thereof.

Also provided herein are methods of killing a senescent cell, the method comprising contacting the senescent cell with an effective amount of a compound of the formula:

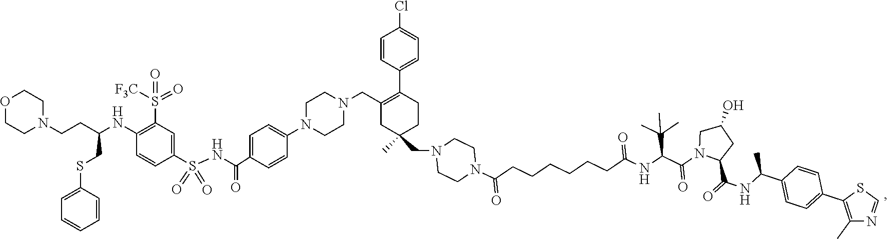 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt thereof, or a pharmaceutical composition thereof.

In certain embodiments, the compound, or a pharmaceutically acceptable salt, solvate, hydrate, polymorph, co-crystal, tautomer, stereoisomer, isotopically labeled compound, or prodrug thereof, or a pharmaceutical composition thereof reduces the viability of senescent cells by at least 5%, at least 10%, at least 15%, at least 20%, at least 25%, at least 30%, at least 40%, or at least 50% at a concentration of 100 nM or less. In some embodiments, the compound reduces the viability of senescent cells by at least 5%, at least 10%, at least 15%, at least 20%, at least 25%, at least 30%, at least 40%, at least 50%, at least 60%, at least 70%, at least 75%, at least 80%, at least 85%, at least 90%, at least 95%, at least 97%, or at least 99% at a compound concentration of 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less. In some embodiments, the compound reduces the viability of senescent cells by at least 5%, at least 10%, at least 15%, at least 20%, at least 25%, at least 30%, at least 40%, or at least 50% at a compound concentration of 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less. In some embodiments, the compound reduces the viability of senescent cells by at least 5%, at least 10%, at least 15%, at least 20%, at least 25%, at least 30%, at least 40%, at least 50%, at least 60%, at least 70%, at least 75%, at least 80%, at least 85%, at least 90%, at least 95%, at least 97%, or at least 99% at a compound concentration of 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less.

In some embodiments, the compound does not significantly reduce the viability of non-senescent cells. In some embodiments, the compound reduces the viability of a non-senescent cell by less than 25%, less than 20%, less than 15%, less than 10%, less than 5%, less than 2%, less than 1%, or less than 0.5% at a compound concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less.

In some embodiments, the compound reduces the viability of platelets by less than 50%, less than 40%, less than 30%, less than 25%, less than 20%, less than 15%, less than 10%, less than 5%, less than 2%, less than 1%, or less than 0.5% at a compound concentration of 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less.

In some embodiments, the compound kills senescent cells in the liver and the spleen. In some embodiments, the compound kills senescent cells in the liver. In some embodiments, the compound kills senescent cells in the spleen. In some embodiments, the compound selectively kills senescent cells in the liver and/or spleen over one or more of the lungs, the kidneys, or fat tissue.

In some embodiments, the compound reduces expression of a senescent marker. In some embodiments, the compound reduces expression of a senescent marker in liver and/or spleen tissue. In some embodiments, the compound reduces expression of Cdkn2a (p16) mRNA in liver and/or spleen tissue. In some embodiments, the compound reduces expression of Cdkn2a (p16) mRNA in liver tissue. In some embodiments, the compound reduces expression of Cdkn2a (p16) mRNA in spleen tissue, In some embodiments, the compound reduces expression of Cdkn2a (p16) mRNA in liver and/or spleen tissue by about 95%, about 90%, about 80%, about 70%, about 60%, about 50%, about 40%, about 30%, about 20%, or about 10%. In some embodiments, the compound reduces expression of Cdkn2a (p16) mRNA in liver tissue by about 95%, about 90%, about 80%, about 70%, about 60%, about 50%, or about 40%. In some embodiments, the compound reduces expression of Cdkn2a (p16) mRNA in spleen tissue by about 95%, about 90%, about 80%, about 70%, about 60%, about 50%, or about 40%. In some embodiments, the compound selectively reduces expression of Cdkn2a (p16) mRNA in liver and/or spleen tissue over one or more of lung tissue, kidney tissue, or fat tissue.

In some embodiments, the compound inhibits upregulation of a senescent marker. In some embodiments, the compound inhibits upregulation of a senescent marker in liver and/or spleen tissue. In some embodiments, the compound inhibits upregulation of p16 mRNA levels. In some embodiments, the compound inhibits upregulation of p21 mRNA levels. In some embodiments, the compound inhibits upregulation of p16 mRNA levels in the liver. In some embodiments, the compound inhibits upregulation of p21 mRNA levels in the liver. In some embodiments, the compound inhibits upregulation of p16 mRNA levels by about 10%, about 20%, about 30%, about 40%, about 50%, about 60%, about 70%, about 80%, or about 90%. In some embodiments, the compound inhibits upregulation of p21 mRNA levels by about 10%, about 20%, about 30%, about 40%, about 50%, about 60%, about 70%, about 80%, or about 90%.

In some embodiments, the contacting is in vitro or in vivo. In certain embodiments, the contacting is in vitro. In certain embodiments, in vitro methods provided herein can be carried out, e.g., in an assay, cell culture, or biological sample. In some embodiments, the contacting is in vivo, e.g., in an organism. In some embodiments, any of the compounds or compositions described herein are contacted with a cell ex vivo, meaning the cell is removed from an organism prior to the contacting. As will be evident to one of skill in the art, the term cell may be used to refer to a single cell as well as a population of cells. In some embodiments, the populations cells are contacted with any of the compounds described herein to regenerate or differentiate one or more cells in the population of cells. In some embodiments, the populations cells are contacted with any of the compounds described herein for use in personalized medicine, for example for diagnostic and/or therapeutic purposes.

In some embodiments, the methods further comprise measuring or assessing the level of one or more properties of the cell. In some embodiments, the level of one or more properties of the cell is assessed following contacting the cell with any of the compounds or compositions described herein. In some embodiments, the level of one or more properties following contacting the cell with any of the compounds or compositions described herein is compared to the level of one or more properties in a reference sample or prior to contacting the cell with the compounds or composition. In some embodiments, the contacting the cell with any of the compounds or compositions described herein increases one or more properties of the cell. In some aspects, the methods described herein may be used to determine whether a cell is susceptible to treatment with the compounds or compositions described herein. In some embodiments, if the level of one or more properties is increased following contacting the cell with any of the compounds or compositions described herein, the cell is determined to be susceptible to treatment with the compound or composition. In some embodiments, if the level of one or more properties is increased following contacting the cell with any of the compounds or compositions described herein, the compound or composition is determined to be a candidate for a disease or disorder associated with the cell.

In certain embodiments, the senescent cells are liver cells or spleen cells. In some embodiments, the senescent cells are liver cells. In certain embodiments, the senescent cells are spleen cells. In some embodiments, the senescent cell is an ionizing radiation-induced senescent cell. In certain embodiments, the senescent cell is an ionizing radiation-induced senescent WI-38 fibroblast cell, ionizing radiation induced senescent human umbilical vein endothelial cell, or ionizing radiation-induced replicative senescent renal epithelial cell. In some embodiments, the senescent cell is an ionizing radiation-induced senescent WI-38 fibroblast cell. In certain embodiments, the senescent cell is an ionizing radiation induced senescent human umbilical vein endothelial cell. In some embodiments, the senescent cell is an ionizing radiation-induced replicative senescent renal epithelial cell.

Any of the methods provided herein may further comprise degrading Bcl-2 and/or Bcl-xl proteins. In certain embodiments, the method comprises degrading Bcl-2. In some embodiments, the method comprises degrading Bcl-xl proteins. In some embodiments, the degrading of Bcl-2 and/or Bcl-xl is achieved in senescent cells.

The methods and uses disclosed herein involve administering any of the compounds or compositions described herein to a subject. In some embodiments, the subject is identified as being in need of treatment. In certain embodiments the subject is suffering from a disease associated with senescent cell accumulation (e.g., age-related disease and/or liver disease). In some embodiments, the subject is susceptible to a disease associated with senescent cell accumulation (e.g., age-related disease and/or liver disease). In some embodiments, the subject is diagnosed with a disease associated with senescent cell accumulation (e.g., age-related disease and/or liver disease).

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In some embodiments, the subject is not suffering from cancer. In certain embodiments, the subject is not suffering from solid tumor cancer.

Any of the methods provided herein may comprise administering an effective amount of a compound or composition provided herein. In certain embodiments, the methods provided herein comprise administering an effective amount of a compound of the formula:

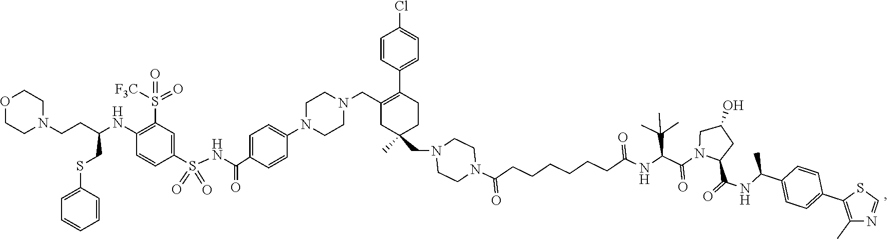 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt thereof, or a pharmaceutical composition thereof.

In certain embodiments, the effective amount is a therapeutically effective amount. In certain embodiments, a therapeutically effective amount is an amount sufficient for treating a disease or disorder (e.g., a disease associated with senescent cell accumulation (e.g., age-related disease and/or liver disease)). In certain embodiments, a therapeutically effective amount is an amount sufficient for ameliorating insulin resistance. In certain embodiments, a therapeutically effective amount is an amount sufficient for ameliorating pre-diabetes or diabetes. In certain embodiments, a therapeutically effective amount is an amount sufficient for killing a senescent cell. In certain embodiments, a therapeutically effective amount is an amount sufficient for killing a senescent cell and treating a disease or disorder (e.g., a disease associated with senescent cell accumulation (e.g., age-related disease and/or liver disease)). In certain embodiments, a therapeutically effective amount is an amount sufficient for killing a senescent cell and ameliorating insulin resistance. In certain embodiments, a therapeutically effective amount is an amount sufficient for killing a senescent cell and ameliorating pre-diabetes or diabetes. In certain embodiments, a therapeutically effective amount is an amount sufficient for degrading Bcl-2 and/or Bcl-xl proteins. In certain embodiments, a therapeutically effective amount is an amount sufficient for degrading Bcl-2 and/or Bcl-xl proteins and treating a disease or disorder (e.g., a disease associated with senescent cell accumulation (e.g., age-related disease and/or liver disease)). In certain embodiments, a therapeutically effective amount is an amount sufficient for degrading Bcl-2 and/or Bcl-xl proteins and ameliorating insulin resistance. In certain embodiments, a therapeutically effective amount is an amount sufficient for degrading Bcl-2 and/or Bcl-xl proteins and ameliorating pre-diabetes or diabetes.

In certain embodiments, the effective amount is a prophylactically effective amount. In certain embodiments, a prophylactically effective amount is an amount sufficient for preventing a disease or disorder (e.g., a disease associated with senescent cell accumulation (e.g., age-related disease and/or liver disease)). In certain embodiments, a prophylactically effective amount is an amount sufficient for killing a senescent cell. In certain embodiments, a prophylactically effective amount is an amount sufficient for killing a senescent cell and preventing a disease or disorder (e.g., a disease associated with senescent cell accumulation (e.g., age-related disease and/or liver disease)). In certain embodiments, a prophylactically effective amount is an amount sufficient for killing a senescent cell and preventing insulin resistance. In certain embodiments, a prophylactically effective amount is an amount sufficient for killing a senescent cell and preventing pre-diabetes or diabetes. In certain embodiments, a prophylactically effective amount is an amount sufficient for degrading Bcl-2 and/or Bcl-xl proteins. In certain embodiments, a prophylactically effective amount is an amount sufficient for degrading Bcl-2 and/or Bcl-xl proteins and preventing a disease or disorder (e.g., a disease associated with senescent cell accumulation (e.g., age-related disease and/or liver disease)). In certain embodiments, a prophylactically effective amount is an amount sufficient for degrading Bcl-2 and/or Bcl-xl proteins and preventing insulin resistance. In certain embodiments, a prophylactically effective amount is an amount sufficient for degrading Bcl-2 and/or Bcl-xl proteins and preventing pre-diabetes or diabetes.

The compounds or compositions provided herein may be administered in an effective amount via a dose. Dosage may be adjusted appropriately to achieve a desired local level of the compound. The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound provided herein.

In some embodiments, the effective amount of the compound is about between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive. In certain embodiments, the effective amount includes independently between 1 mg and 3 mg, inclusive, of a compound described herein. In certain embodiments, the effective amount described herein includes independently between 3 mg and 10 mg, inclusive, of a compound described herein. In certain embodiments, the effective amount described herein includes independently between 10 mg and 30 mg, inclusive, of a compound described herein. In certain embodiments, the effective amount includes independently between 30 mg and 100 mg, inclusive, of a compound described herein.

In some embodiments, the effective amount has a concentration of the compound of about between 0.001 μM and 0.01 μM, 0.01 μM and 0.1 μM, between 0.1 μM and 1 μM, between 1 μM and 10 μM, between 10 μM and 50 μM, or between 50 μM and 100 μM, inclusive. In certain embodiments, the effective amount includes independently between 0.01 0.01 μM and 0.1 μM, inclusive, of a compound described herein. In certain embodiments, the effective amount includes independently between 1 μM and 3 μM, inclusive, of a compound described herein. In certain embodiments, the effective amount includes independently between 1 μM and 10 μM, inclusive, of a compound described herein. In certain embodiments, the effective amount includes independently between 10 μM and 100 μM, inclusive, of a compound described herein. In certain embodiments, the effective amount includes independently between 0.01 μM and 10 μM, inclusive, of a compound described herein. In certain embodiments, the effective amount includes independently between 0.01 μM and 1 μM, inclusive, of a compound described herein.

In certain embodiments, the effective amount is about 0.1 mg/kg to about 25 mg/kg of the compound. In some embodiments, the effective amount is about 0.1 mg/kg to about 20 mg/kg of the compound. In certain embodiments, the effective amount is about 0.1 mg/kg to about 15 mg/kg of the compound. In some embodiments, the effective amount is about 0.1 mg/kg to about 10 mg/kg of the compound. In certain embodiments, the effective amount is about 0.1 mg/kg to about 5 mg/kg of the compound. In some embodiments, the effective amount is about 1 mg/kg to about 25 mg/kg of the compound. In certain embodiments, the effective amount is about 1 mg/kg to about 20 mg/kg of the compound. In some embodiments, the effective amount is about 1 mg/kg to about 15 mg/kg of the compound. In certain embodiments, the effective amount is about 1 mg/kg to about 10 mg/kg of the compound. In some embodiments, the effective amount is about 1 mg/kg to about 5 mg/kg of the compound. In certain embodiments, the effective amount is about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, or about 25 mg/kg of the compound.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

In some embodiments, the subject is administered an initial dose of any of the compounds or compositions described herein, followed by one or more additional doses of any of the compounds or compositions described herein. In some embodiments, the initial dose may contain a different amount of any of the compounds described herein as compared to the one or more additional doses. In some embodiments, the initial dose is a higher dose (e.g., contains more of any one of the compounds described herein) as compared to the one or more additional doses.

In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

In some embodiments, a compound or composition, as described herein, is administered in combination with one or more additional pharmaceutical agents (e.g., therapeutically and/or prophylactically active agents). In certain embodiments, the compounds or compositions are administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in degrading Bcl-xl and/or Bcl-2 in a subject or cell), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects.

### Pharmaceutical Compositions

In some aspects, the present disclosure provides compositions comprising one or more active ingredients. The present disclosure provides pharmaceutical compositions comprising a compound of the formula:

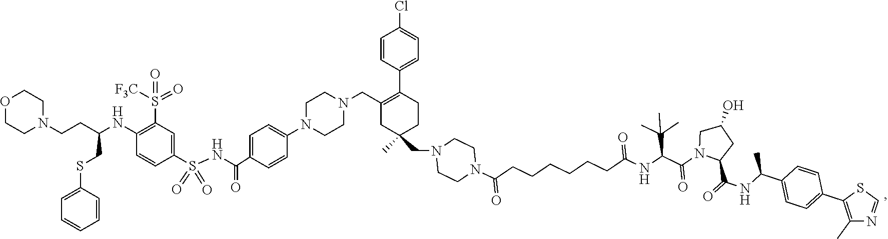 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt, hydrate, solvate, or prodrug thereof. In some embodiments, the pharmaceutical composition comprises a pharmaceutically acceptable salt of the compound. In certain embodiments, the pharmaceutical composition further comprises a pharmaceutically acceptable excipient.

In certain embodiments, the compound described herein is provided in an effective amount in the pharmaceutical composition.

In certain embodiments, the effective amount is a therapeutically effective amount. In certain embodiments, a therapeutically effective amount is an amount sufficient for treating a disease or disorder (e.g., a disease associated with senescent cell accumulation (e.g., age-related disease and/or liver disease)). In certain embodiments, a therapeutically effective amount is an amount sufficient for ameliorating insulin resistance. In certain embodiments, a therapeutically effective amount is an amount sufficient for ameliorating pre-diabetes or diabetes. In certain embodiments, a therapeutically effective amount is an amount sufficient for killing a senescent cell. In certain embodiments, a therapeutically effective amount is an amount sufficient for killing a senescent cell and treating a disease or disorder (e.g., a disease associated with senescent cell accumulation (e.g., age-related disease and/or liver disease)). In certain embodiments, a therapeutically effective amount is an amount sufficient for killing a senescent cell and ameliorating insulin resistance. In certain embodiments, a therapeutically effective amount is an amount sufficient for killing a senescent cell and ameliorating pre-diabetes or diabetes. In certain embodiments, a therapeutically effective amount is an amount sufficient for degrading Bcl-2 and/or Bcl-xl proteins. In certain embodiments, a therapeutically effective amount is an amount sufficient for degrading Bcl-2 and/or Bcl-xl proteins and treating a disease or disorder (e.g., a disease associated with senescent cell accumulation (e.g., age-related disease and/or liver disease)). In certain embodiments, a therapeutically effective amount is an amount sufficient for degrading Bcl-2 and/or Bcl-xl proteins and ameliorating insulin resistance. In certain embodiments, a therapeutically effective amount is an amount sufficient for degrading Bcl-2 and/or Bcl-xl proteins and ameliorating pre-diabetes or diabetes.

In certain embodiments, the effective amount is a prophylactically effective amount. In certain embodiments, a prophylactically effective amount is an amount sufficient for preventing a disease or disorder (e.g., a disease associated with senescent cell accumulation (e.g., age-related disease and/or liver disease)). In certain embodiments, a prophylactically effective amount is an amount sufficient for killing a senescent cell. In certain embodiments, a prophylactically effective amount is an amount sufficient for killing a senescent cell and preventing a disease or disorder (e.g., a disease associated with senescent cell accumulation (e.g., age-related disease and/or liver disease)). In certain embodiments, a prophylactically effective amount is an amount sufficient for killing a senescent cell and preventing insulin resistance. In certain embodiments, a prophylactically effective amount is an amount sufficient for killing a senescent cell and preventing pre-diabetes or diabetes. In certain embodiments, a prophylactically effective amount is an amount sufficient for degrading Bcl-2 and/or Bcl-xl proteins. In certain embodiments, a prophylactically effective amount is an amount sufficient for degrading Bcl-2 and/or Bcl-xl proteins and preventing a disease or disorder (e.g., a disease associated with senescent cell accumulation (e.g., age-related disease and/or liver disease)). In certain embodiments, a prophylactically effective amount is an amount sufficient for degrading Bcl-2 and/or Bcl-xl proteins and preventing insulin resistance. In certain embodiments, a prophylactically effective amount is an amount sufficient for degrading Bcl-2 and/or Bcl-xl proteins and preventing pre-diabetes or diabetes.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

In certain embodiments, the compound or composition further comprises one or more additional agents. In some embodiments, the compound or composition is administered concurrently with, prior to, or subsequent to one or more additional pharmaceutical agents. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins.

In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

### Kits

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

In another aspect, the present disclosure provides a kit comprising:
a first container comprising a compound of the formula:

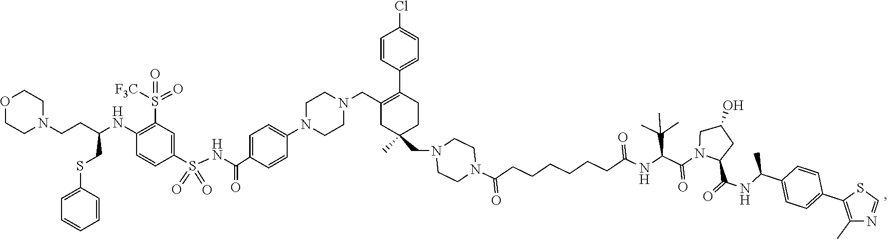 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt, solvate, hydrate, polymorph, co-crystal, tautomer, stereoisomer, isotopically labeled compound, or prodrug thereof, or a pharmaceutical composition thereof; and
instructions for using the compound, or a pharmaceutically acceptable salt, solvate, hydrate, polymorph, co-crystal, tautomer, stereoisomer, isotopically labeled compound, or prodrug thereof, or a pharmaceutical composition thereof, in a method provided herein.

Thus, in one aspect, provided are kits including a first container comprising a compound or pharmaceutical composition described herein. In certain embodiments, the kits are useful for treating a disease or disorder (e.g., a disease associated with senescent cell accumulation (e.g., age-related disease and/or liver disease)) in a subject in need thereof. In certain embodiments, the kits are useful for preventing a disease or disorder (e.g., a disease associated with senescent cell accumulation (e.g., age-related disease and/or liver disease)) in a subject in need thereof. In certain embodiments, the kits are useful for reducing the risk of developing a disease or disorder (e.g., a disease associated with senescent cell accumulation (e.g., age-related disease and/or liver disease)) in a subject in need thereof.

In certain embodiments, a kit described herein further includes instructions for using the kit. In some embodiments, a kit disclosed herein includes information as required by a regulatory agency such as the U.S. Food and Drug Administration (FDA). In certain embodiments, the information included in the kits is prescribing information. In certain embodiments, the kits and instructions provide for treating a disease in a subject in need thereof. In certain embodiments, the kits and instructions provide for preventing a disease or disorder (e.g., a disease associated with senescent cell accumulation (e.g., age-related disease and/or liver disease)) in a subject in need thereof. In certain embodiments, the kits and instructions provide for reducing the risk of developing a disease or disorder (e.g., a disease associated with senescent cell accumulation (e.g., age-related disease and/or liver disease)) in a subject in need thereof. A kit described herein may include one or more additional pharmaceutical agents described herein as a separate composition.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### Example 1. Development of Novel Bcl-xl and Bcl-2 Dual PROTACs/Degraders

ABT263 (also known as navitoclax), a B-cell lymphoma-2 (Bcl-2) and B-cell lymphoma-extra large (Bcl-xl) dual inhibitor^{23,24}, is one of the most potent and broad-spectrum senolytic agents identified to date^{16,17,18}. Bcl-xl inhibition with ABT263 and other small molecular inhibitors induces platelet apoptosis and results in severe thrombocytopenia, which prevents the use of ABT263 and other Bcl-xl specific inhibitors in the clinic-even for cancer patients-because platelets solely depend on Bcl-xl for survival^{24,25,26,27}. By contrast, Bcl-2 is dispensable for thrombopoiesis and platelet survival in mice and humans²⁸ and inhibition of Bcl-2 with ABT199 (also known as venetoclax) does not induce thrombocytopenia²⁷. ABT263 on-target toxicity can be reduced to generate a safer senolytic agent by converting ABT263 into a platelet-sparing Bcl-xl proteolysis-targeting chimera (PROTAC).

PROTACs are bivalent small molecules containing a ligand that recognizes a target protein linked to another ligand that recruits a specific E3 ubiquitin ligase^{29,30}. PROTAC binding induces proximity-induced ubiquitination of the target protein and its subsequent degradation by proteasomes. Thus, PROTACs act catalytically to induce protein degradation in a sub-stoichiometric manner. Since their effect is not limited by equilibrium occupancy, it results in less drug exposure and reduced toxicity compared with traditional inhibitors. Because of their improved and prolonged activity profile, they are increasingly used to develop more effective antitumor agents and other therapeutics^{31,32,33}. Importantly, because PROTACs rely on E3 ligases to induce protein degradation, it is possible to achieve cell/tissue selectivity, even when the target proteins are ubiquitously expressed as long as they target the proteins to an E3 ligase that is cell- or tissue-specific.

PROTAC technology has been used to reduce ABT263 on-target toxicity by converting ABT263 into PZ15227, a Bcl-xl specific PROTAC (Bcl-xl-P), which targets Bcl-xl to the E3 ligase cereblon (CRBN) that is poorly expressed in platelets^{34,35,36}. PZ15227 has the chemical structure:

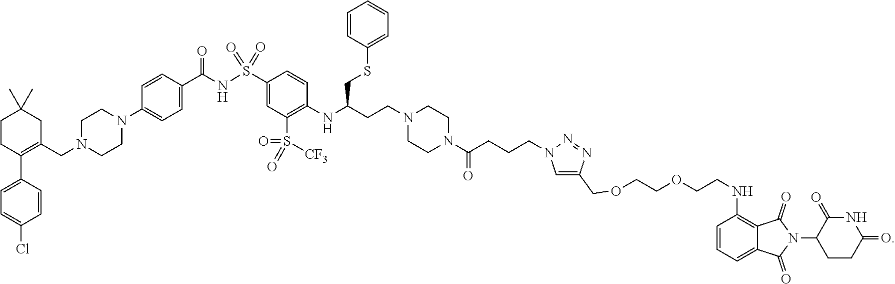 [SMILES: CC1(C)CCC(c2ccc(Cl)cc2)=C(CN2CCN(c3ccc(C(=O)NS(=O)(=O)c4ccc(N[C@H](CCN5CCN(C(=O)CCCn6cc(COCCOCCNc7cccc8c7C(=O)N(C7CCC(=O)NC7=O)C8=O)nn6)CC5)CSc5ccccc5)c(S(=O)(=O)C(F)(F)F)c4)cc3)CC2)C1]

It was found that PZ15227 is less toxic to platelets but equally or slightly more potent against SnCs compared with ABT263. These findings provide an approach to reduce on-target toxicity of toxic senolytic agents. With further improvement, Bcl-xl-Ps have the potential to be developed into safer and more effective senolytics than ABT263.

Novel Bcl-xl and Bcl-2 dual PROTACs/degraders have been developed by using one of the two methyl groups on the cyclohexene ring of ABT263 as the linker attachment site to link ABT263 to the VHL ligand to target Bcl-xl and Bcl-2 to VHL for ubiquitination and proteasome degradation. These Bcl-xl and Bcl-2 dual PROTACs/degraders are not only potent antitumor agents but also effective senolytic agents. They have the potential to clear senescent cells (SnCs) to prevent and treat age-related diseases and diseases caused by SnC induction and accumulation, such as alcohol-induced liver diseases and nonalcoholic fatty liver diseases ranging from non-alcoholic steatohepatitis (NASH), cirrhosis, and hepatocellular carcinoma (HCC). In particular, 753b has been developed as a Bcl-xl/Bcl-2 dual PROTAC/degrader. Compound 753b has the chemical structure:

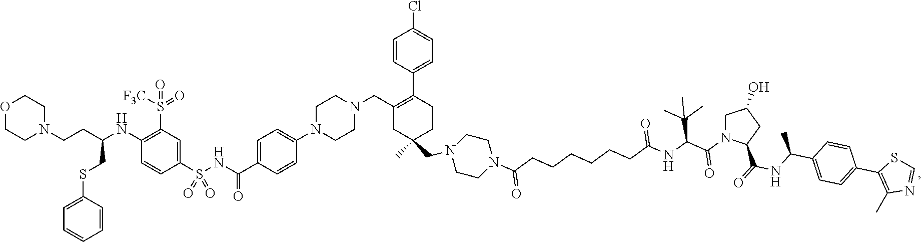 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

### Example 2. 753b is a Bcl-Xl and Bcl-2 Dual PROTAC/Degrader

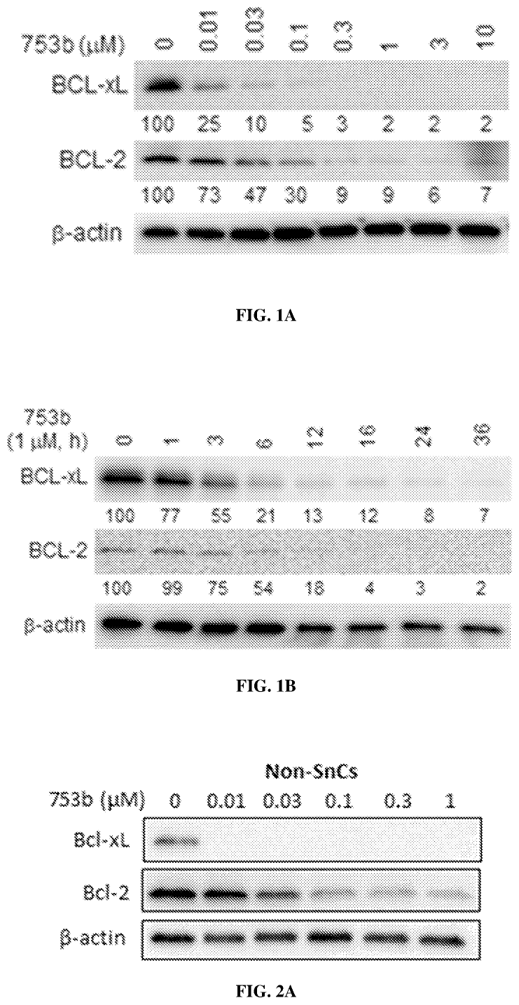

753b degrades Bcl-xl and Bcl-2 in a dose- and time-dependent manner in HEK293T cells. The potency of 753b in degrading Bcl-xl/2 was evaluated in 293T cells by immunoblots after the cells were treated with different concentrations of 753b for 16 h (FIG. 1A) or after the cells were treated with 1 μM 753b for various time points as indicated (FIG. 1B). Representative immunoblots are shown, and β-actin was used as a loading control.

### Example 3. 753b is a Better Senolytic Agent than ABT263

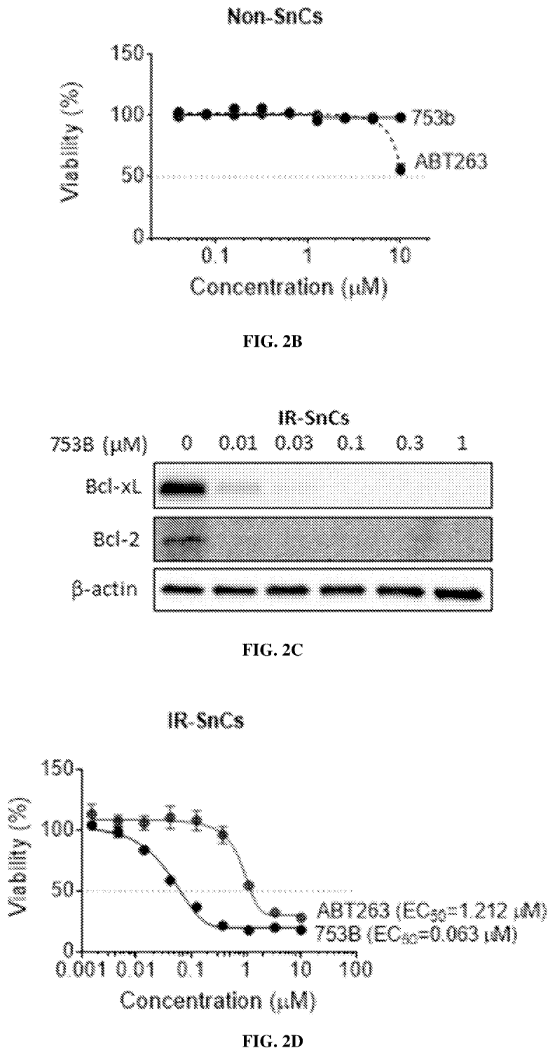

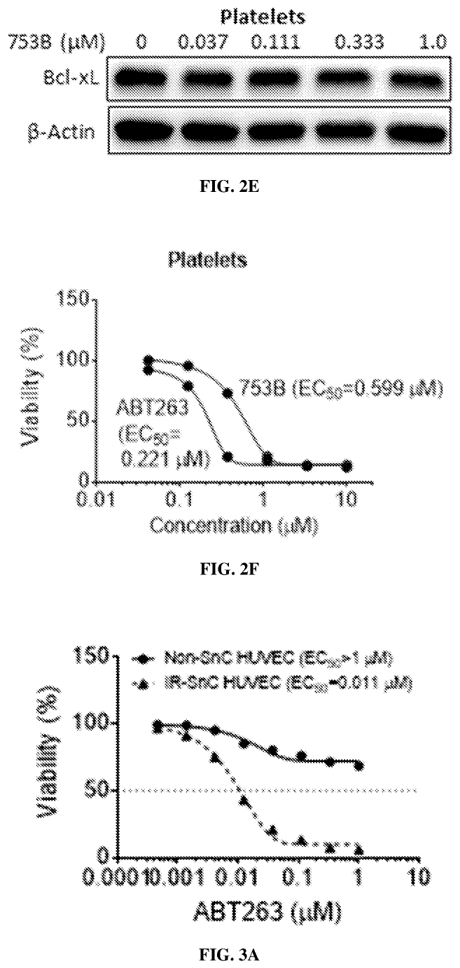

753b is a more potent senolytic agent against ionizing radiation (IR) induced senescent WI-38 fibroblast cells (IR-SnCs) but is less toxic to normal non-senescent WI-38 fibroblast cells (Non-SnCs) and human platelets than ABT263 (FIGS. 2A, 2C, 2E). 753b degrades Bcl-xl and Bcl-2 in Non-SnCs and IR-SnCs, but not in human platelets, in a dose-dependent manner. The potency of 753b in degrading Bcl-xl/2 was evaluated by immunoblots after the cells or platelets were treated with different concentrations of 753b for 16 h (FIGS. 2B, 2D, 2F). 753b is more cytotoxic to IR-SnCs but less toxic to Non-SnCs and platelets. The viability of Non-SnCs, IR-SnCs and platelets was measured 72 hr (Non-SnCs and IR-SnCs) and 48 hr (platelets) after they were incubated with increasing concentrations of 753b or ABT263 compared to cells/platelets with vehicle.

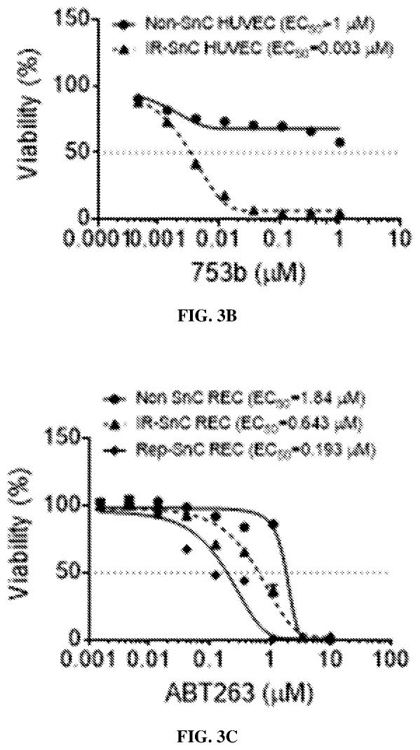

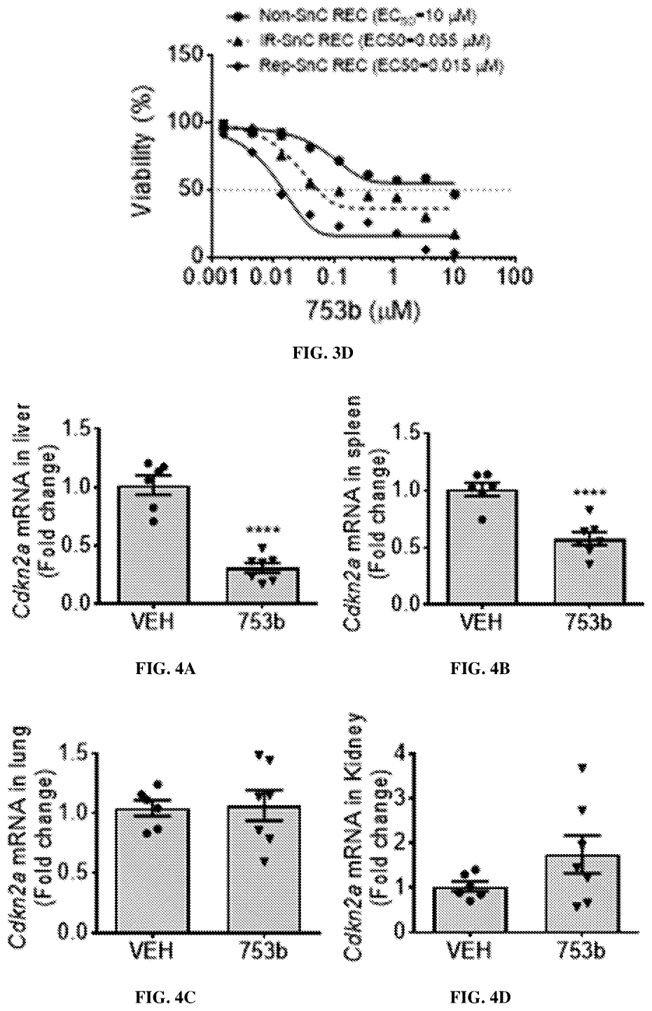

753b is a more potent senolytic agent against IR-induced senescent human umbilical vein endothelial cells (IR-SnC HUVEC) and renal epithelial cells (IR-SnC REC) and replicative senescent REC (Rep-SnC REC) but less toxic to normal non-senescent HUVEC (Non-SnC HUVEC) and REC (Non-SnC REC) than ABT263 (FIGS. 3A to 3D). The viability of Non-SnC HUVEC, Non-SnC REC, IR-SnC HUVEC, IR-SnC REC, and Rep-SnC REC was measured 72 hr after they were incubated with increasing concentrations of 753b or ABT263 compared to cells/platelets with vehicle.

### Example 4. 753b is a Potent Senolytic Agent that can Selectively Clear SnCs in the Liver and Spleen in Naturally Aged Mice

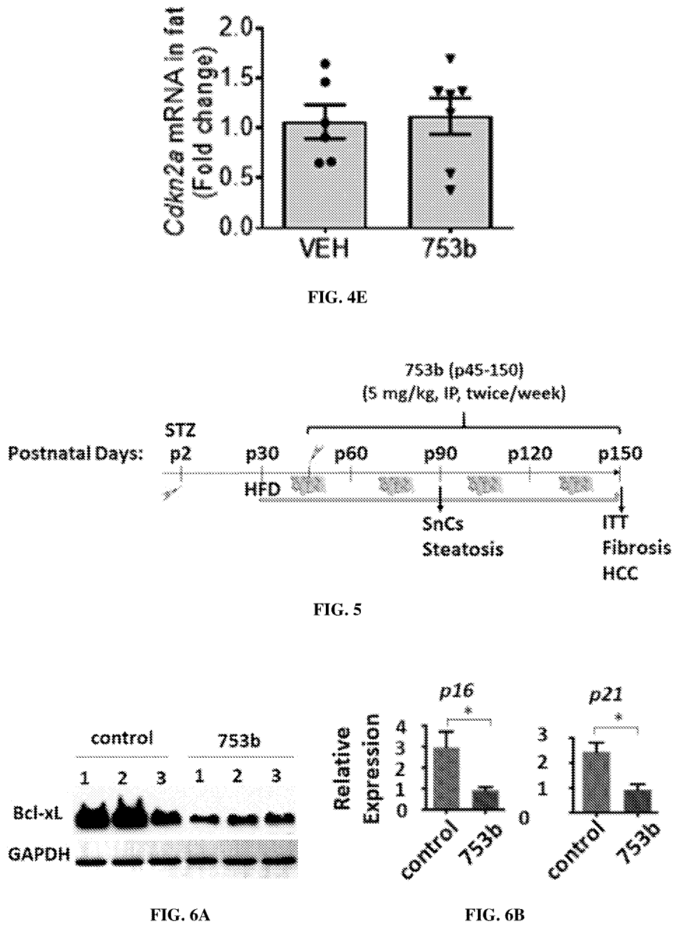

753b can selectively clear SnCs in the liver and spleen in naturally aged mice. Aged (20 months old) C₅₇BL/6 mice were treated with vehicle (n=6) or 753b (5 mg/kg, q3d for 8 times, ip; n=7). The livers, spleens, lungs, kidneys and inguinal fat tissues were harvested after the mice were euthanized. The expression of Cdkn2a (p16) mRNA in these tissues was analyzed by qPCR and presented as mean+/−SE of fold changes compared to that in tissues from vehicle-treated mice (FIGS. 4A to 4E).

### Example 5. 753b can Inhibit High Fat Diet (HFD)-Induced Nonalcoholic Fatty Liver Diseases, Including NASH, Cirrhosis and HCC

Experimental design: C₅₇BL6 mice were injected with Streptozotocin (STZ) at postnatal day 2 to induce diabetes and then fed with HFD from postnatal day 30 till the end of the experiments (FIG. 5). Half of the mice started receiving vehicle and the others receiving 753b (5 mg/kg) treatment on postnatal day 45 twice a week by IP injection till the end of the experiments. A group of the mice were euthanized at postnatal day 90 (or day 45 after the initiation of the treatment) for analysis of senescent cell accumulation and steatosis. Another group of the mice were euthanized at postnatal day 150 (or day 105 after the initiation of the treatment) for analysis of liver fibrosis and cancer.

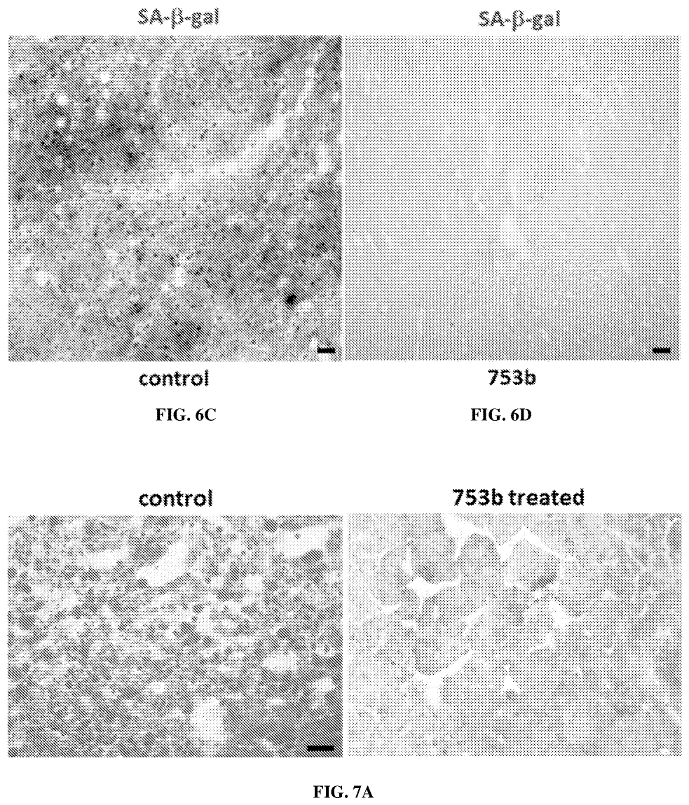

753b effectively clears senescent cells induced by STZ/HFD in the liver. The liver tissues were collected from mice at postnatal day 90 (p90 or 45 days after treatment) for the analyses. Western blotting showed that 753b administration reduced Bcl-xL accumulation in the livers of STZ/HFD-treated mice compared to vehicle controls (FIG. 6A). Q-RT-PCR detected that 753b administration inhibited upregulation of senescence markers p16 and p21 at mRNA levels (FIG. 6B). Data were means±SD of triplicate experiments. Measurement of SA-beta-galactosidase (SA-β-gal) activity by SA-β-gal staining showed 753b administration reduced senescent cell burden in the STZ/HFD-treated livers (FIG. 6C).

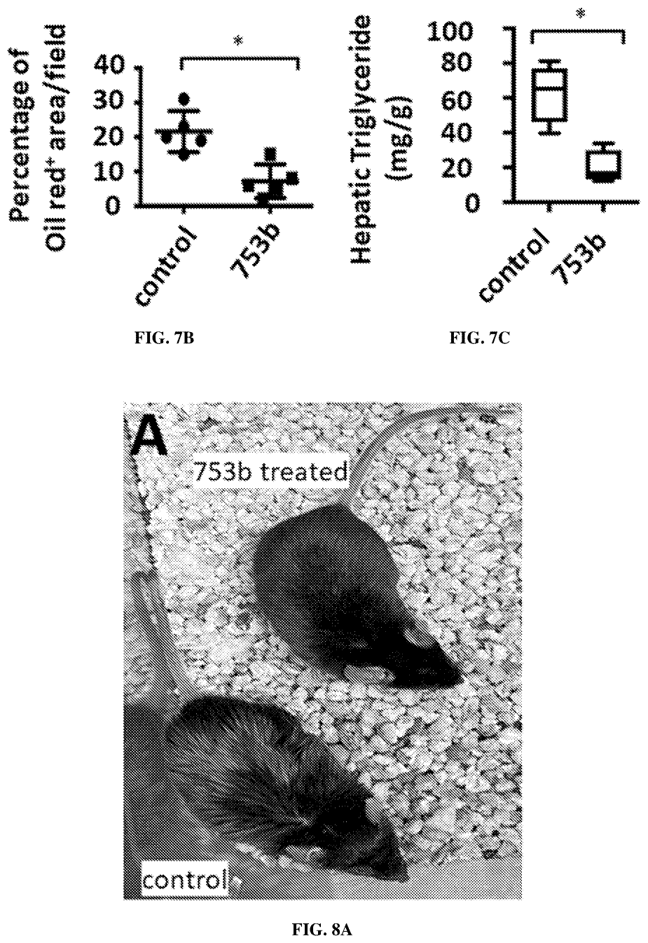

753b administration represses hepatic fat accumulation in the STAM model. The liver tissues were collected from mice at postnatal day 90 (p90 or 45 days after treatment) for the analyses. Representative images for oil red staining showed that 753b administration inhibited hepatic fat accumulation (FIG. 7A). Quantification of oil red stained images was performed from 5 livers per group (FIG. 7B). 753b administration decreased levels of hepatic triglyceride (FIG. 7C). Quantification was performed from 5 livers per group and was expressed as means±SD.

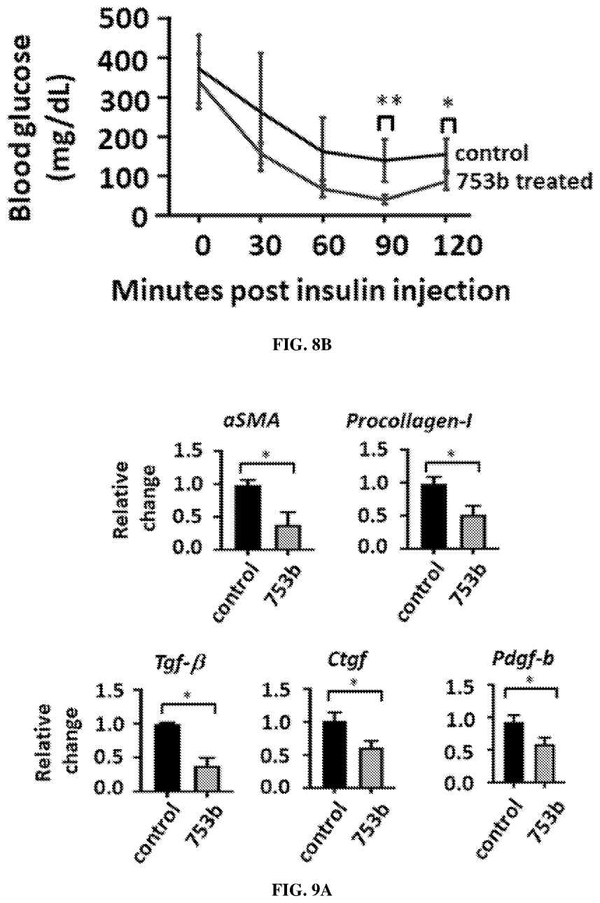

753b administration improves body conditions and ameliorates the development of insulin resistance in the STAM model. All the analyses were done in mice at postnatal day 150 (p150 or 105 days after treatment). Morphological visualization indicated that 753b administration prevented the development of abnormal body conditions (dehydration and dampen hair) in the STZ/HFD-treated mice (FIG. 8A). Intraperitoneal injected insulin tolerance test (ITT) showed that 753b administration prevented insulin resistance (FIG. 8B).

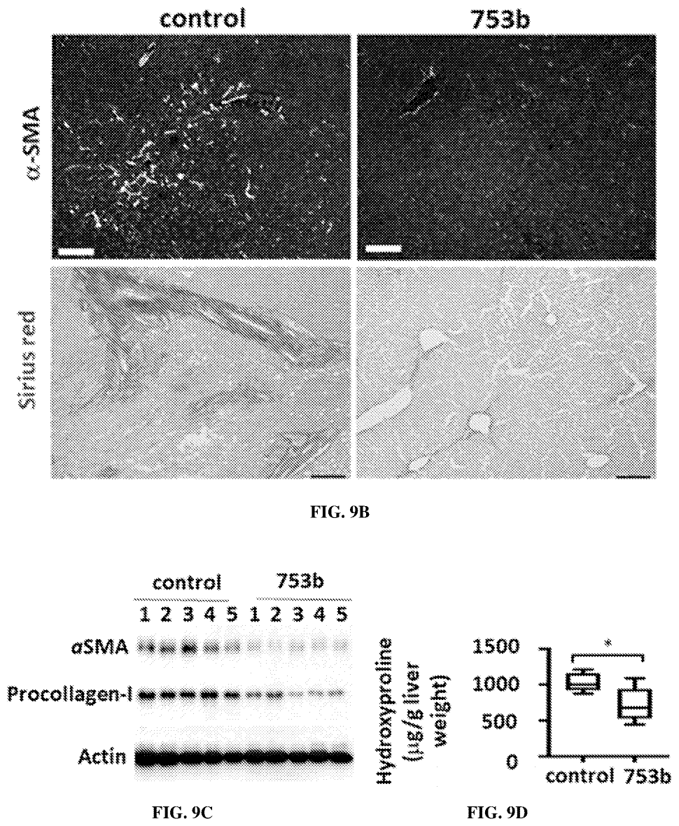

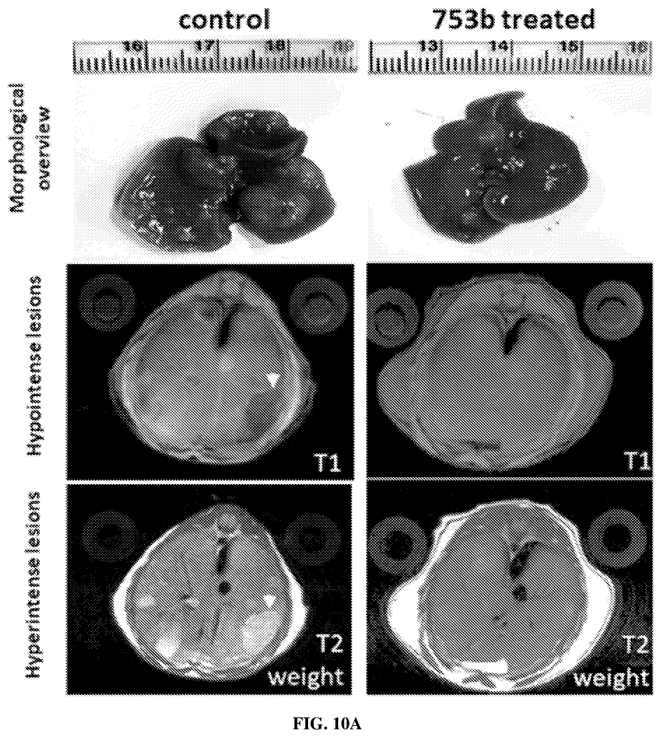

753b administration inhibits myofibroblast cell activation and liver fibrosis in the STAM model. All the analyses were done in mice at postnatal day 150 (p150 or 105 days after treatment). Q-RT-PCR analysis detected lower levels of fibrosis-related genes in livers of STZ/HFD-treated mice that received 753b administration in comparison to controls (FIG. 9A). Immunofluorescent staining of aSMA and sirius red staining for collagen fibril indicated 753b administration prevented liver fibrosis based little activation of myofibroblast cells and decreased collagen deposition in the STZ/HFD-treated livers (FIG. 9B). Western blotting detected low levels of aSMA and pro-collagen type I proteins in the STZ/HFD/753b-treated livers (FIG. 9C). Hepatic hydroxyproline assay confirmed decreased levels of collagen in the STZ/HFD-treated and 753b-administered livers (FIG. 9D). Quantification was performed from 5 livers per group and was expressed as means±SD.

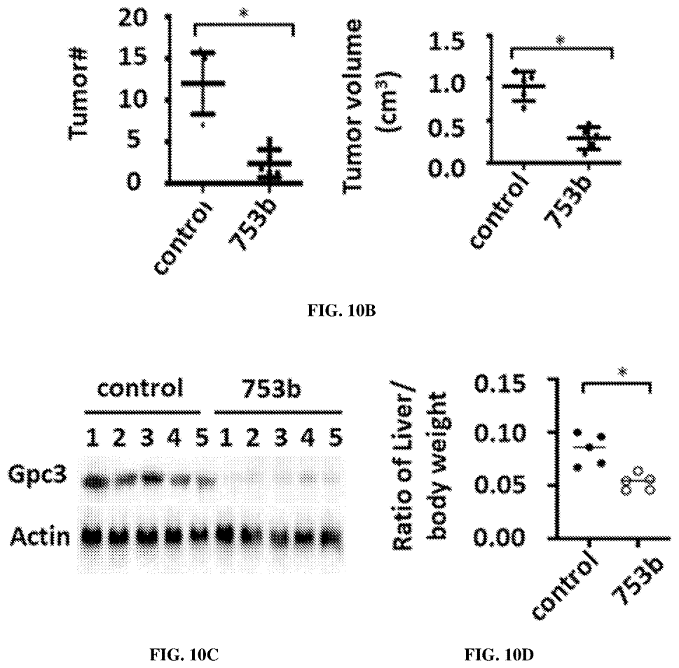

753b administration reduces tumorigenesis in the STAM model. All the analyses were done in mice at postnatal day 150 (p150 or 105 days after treatment). Morphological overview of the STZ/HFD-treated livers in mice that received 753b were compared to mice that received vehicle treatment. MRI of T1 pre-contrast and T2-weighted images reveal differences in the corresponding livers. Tumor lesions were determined based on hypointense signals in T1 but hyperintense signals in T2-weighted image (FIG. 10A). Tumor number and volume was quantified based on MRI T1 and T2-weighted images. Values were expressed as means±SD from 5 mice per group. Data for each mouse were analyzed based on 5-6 slices of images that covered whole liver lobes (FIG. 10B). Western blotting of Gpc3 was performed (FIG. 10C). Quantification analysis was performed for the ratio between liver and body weight in the STZ/HFD treated livers that were exposed to 753b or control compound (FIG. 10D). Values represent means±SD from 5 livers per group.

### REFERENCES

1. Childs, B. G., Durik, M., Baker, D. J. & van Deursen, J. M. Cellular senescence in aging and age-related disease: from mechanisms to therapy. Nat. Med. 21, 1424-1435 (2015).
2. Childs, B. G. et al. Senescent cells: an emerging target for diseases of ageing. Nat Rev Drug Discov 16, 718-735 (2017).
3. Kirkland, J. L. & Tchkonia, T. Cellular Senescence: A Translational Perspective. EBioMedicine 21, 21-28 (2017).
4. Niedernhofer, L. J. & Robbins, P. D. Senotherapeutics for healthy ageing. Nat Rev Drug Discov 17, 377 (2018).
5. Ovadya, Y. & Krizhanovsky, V. Strategies targeting cellular senescence. J. Clin. Invest. 128, 1247-1254 (2018).
6. Muñoz-Espín, D. & Serrano, M. Cellular senescence: from physiology to pathology. Nat. Rev. Mol. Cell Biol. 15, 482-496 (2014).
7. McHugh, D. & Gil, J. Senescence and aging: Causes, consequences, and therapeutic avenues. J. Cell Biol. 217, 65-77 (2018).
8. Zhu, Y. et al. The Achilles' heel of senescent cells: from transcriptome to senolytic drugs. Aging Cell 14, 644-658 (2015).
9. Justice, J. N. et al. Senolytics in idiopathic pulmonary fibrosis: Results from a first-in-human, open-label, pilot study. EBioMedicine (2019) doi:10.1016/j.ebiom.2018.12.052.
10. Yousefzadeh, M. J. et al. Fisetin is a senotherapeutic that extends health and lifespan. EBioMedicine 36, 18-28 (2018).
11. Zhu, Y. et al. New agents that target senescent cells: the flavone, fisetin, and the BCL-XL inhibitors, A1331852 and A1155463. Aging (Albany NY) 9, 955-963 (2017).
12. Zhang, X. et al. Oxidation resistance 1 is a novel senolytic target. Aging Cell e12780 (2018) doi:10.1111/acel.12780.
13. Wang, Y. et al. Discovery of piperlongumine as a potential novel lead for the development of senolytic agents. Aging (Albany NY) 8, 2915-2926 (2016).
14. Li, W., He, Y., Zhang, R., Zheng, G. & Zhou, D. The curcumin analog EF24 is a novel senolytic agent. Aging (Albany NY) (2019) doi:10.18632/aging.101787.
15. Xu, M. et al. Senolytics improve physical function and increase lifespan in old age. Nat. Med. 24, 1246-1256 (2018).
16. Chang, J. et al. Clearance of senescent cells by ABT263 rejuvenates aged hematopoietic stem cells in mice. Nat. Med. 22, 78-83 (2016).
17. Yosef, R. et al. Directed elimination of senescent cells by inhibition of BCL-W and BCL-XL. Nat Commun 7, 11190 (2016).
18. Zhu, Y. et al. Identification of a novel senolytic agent, navitoclax, targeting the Bcl-2 family of anti-apoptotic factors. Aging Cell 15, 428-435 (2016).
19. Fuhrmann-Stroissnigg, H. et al. Identification of HSP90 inhibitors as a novel class of senolytics. Nat Commun 8, 422 (2017).
20. Jeon, O. H. et al. Local clearance of senescent cells attenuates the development of post-traumatic osteoarthritis and creates a pro-regenerative environment. Nat. Med. 23, 775-781 (2017).
21. Baar, M. P. et al. Targeted Apoptosis of Senescent Cells Restores Tissue Homeostasis in Response to Chemotoxicity and Aging. Cell 169, 132-147.e16 (2017).
22. Muñoz-Espín, D. et al. A versatile drug delivery system targeting senescent cells. EMBO Mol Med 10, (2018).
23. Tse, C. et al. ABT-263: a potent and orally bioavailable Bcl-2 family inhibitor. Cancer Res. 68, 3421-3428 (2008).
24. Ashkenazi, A., Fairbrother, W. J., Leverson, J. D. & Souers, A. J. From basic apoptosis discoveries to advanced selective BCL-2 family inhibitors. Nat Rev Drug Discov 16, 273-284 (2017).
25. Leverson, J. D. et al. Exploiting selective BCL-2 family inhibitors to dissect cell survival dependencies and define improved strategies for cancer therapy. Sci Transl Med 7, 279ra40 (2015).
26. Gandhi, L. et al. Phase I study of Navitoclax (ABT-263), a novel Bcl-2 family inhibitor, in patients with small-cell lung cancer and other solid tumors. J. Clin. Oncol. 29, 909-916 (2011).
27. Souers, A. J. et al. ABT-199, a potent and selective BCL-2 inhibitor, achieves antitumor activity while sparing platelets. Nat. Med. 19, 202-208 (2013).
28. Debrincat, M. A. et al. BCL-2 is dispensable for thrombopoiesis and platelet survival. Cell Death Dis 6, e1721 (2015).
29. Toure, M. & Crews, C. M. Small-Molecule PROTACS: New Approaches to Protein Degradation. Angew. Chem. Int. Ed. Engl. 55, 1966-1973 (2016).
30. Lai, A. C. & Crews, C. M. Induced protein degradation: an emerging drug discovery paradigm. Nat Rev Drug Discov 16, 101-114 (2017).
31. Veggiani, G., Gerpe, M. C. R., Sidhu, S. S. & Zhang, W. Emerging drug development technologies targeting ubiquitination for cancer therapeutics. Pharmacol. Ther. 199, 139-154 (2019).
32. Neklesa, T. K., Winkler, J. D. & Crews, C. M. Targeted protein degradation by PROTACs. Pharmacol. Ther. 174, 138-144 (2017).
33. Pettersson, M. & Crews, C. M. PROteolysis TArgeting Chimeras (PROTACs)—Past, present and future. Drug Discov Today Technol 31, 15-27 (2019).
34. Bray, P. F. et al. The complex transcriptional landscape of the anucleate human platelet. BMC Genomics 14, 1 (2013).
35. Kissopoulou, A., Jonasson, J., Lindahl, T. L. & Osman, A. Next generation sequencing analysis of human platelet PolyA+ mRNAs and rRNA-depleted total RNA. PLoS ONE 8, e81809 (2013).
36. He Y, Zhang X, Chang J, Kim H N, Zhang P, Wang Y, Khan S, Liu X, Zhang X, Lv D, Song L, Li W, Thummuri D, Yuan Y, Wiegand J S, Ortiz Y T, Budamagunta V, Elisseeff J H, Campisi J, Almeida M, Zheng G, Zhou D. Using proteolysis-targeting chimera technology to reduce navitoclax platelet toxicity and improve its senolytic activity. Nat Commun. 11, 1996 (2020).

### INCORPORATION BY REFERENCE

The contents of all references (including literature references, issued patents, published patent applications, and co-pending patent applications) cited throughout this application are hereby expressly incorporated herein in their entireties by reference.

### EMBODIMENTS

Embodiment 1. A method of treating and/or preventing a disease or disorder in a subject identified in need thereof, the method comprising administering an effective amount of a compound of the formula:

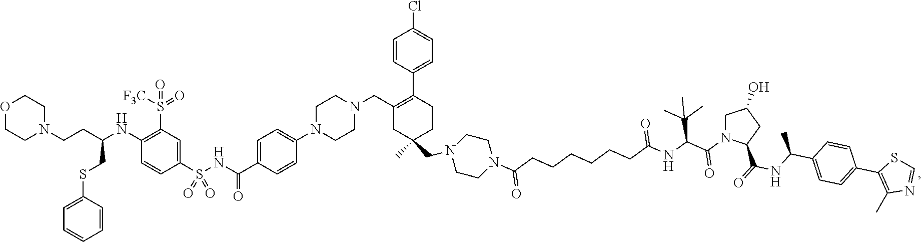 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt thereof, or a pharmaceutical composition thereof.

Embodiment 2. A method of treating a subject suffering from or susceptible to a disease or disorder, the method comprising administering an effective amount of a compound of the formula:

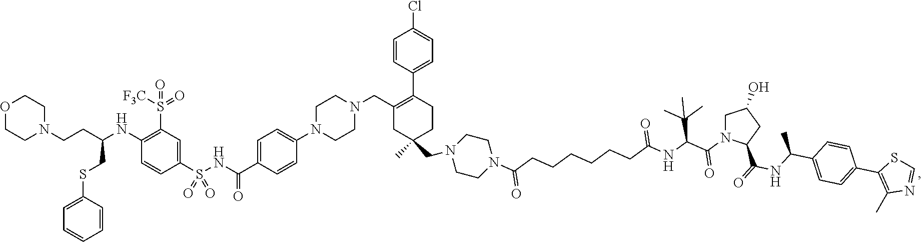 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt thereof, or a pharmaceutical composition thereof.

Embodiment 3. The method of embodiment 1 or 2, wherein the disease is associated with senescent cell accumulation.

Embodiment 4. The method of any of the preceding embodiments, wherein the disease is an age-related disease.

Embodiment 5. The method of any of the preceding embodiments, wherein the disease is a liver disease.

Embodiment 6. The method of any of the preceding embodiments, wherein the disease is an alcohol-induced liver disease.

Embodiment 7. The method of any of the preceding embodiments, wherein the disease is a non-alcoholic fatty liver disease.

Embodiment 8. The method of embodiment 7, wherein the non-alcoholic fatty liver disease is a diabetes- and/or obesity-related non-alcoholic fatty liver disease.

Embodiment 9. The method of any of the preceding embodiments, wherein the disease is liver steatosis, non-alcoholic steatohepatitis (NASH), liver fibrosis, or cirrhosis.

Embodiment 10. The method of embodiment 9, wherein the disease is NASH, liver fibrosis, or cirrhosis.

Embodiment 11. The method of any of the preceding embodiments, wherein the disease is liver cancer.

Embodiment 12. The method of embodiment 11, wherein the liver cancer is hepatocellular carcinoma (HCC).

Embodiment 13. The method of any of the preceding embodiments, wherein the disease is not cancer.

Embodiment 14. The method of embodiment 13, wherein the cancer is a solid tumor.

Embodiment 15. The method of any of the preceding embodiments, wherein the subject is a mammal.

Embodiment 16. The method of embodiment 15, wherein the subject is a human.

Embodiment 17. The method of any of the preceding embodiments, wherein the subject is not suffering from cancer.

Embodiment 18. The method of embodiment 17, wherein the cancer is a solid tumor.

Embodiment 19. The method of any of the preceding embodiments, further comprising suppressing hepatic fat accumulation.

Embodiment 20. The method of embodiment 19, wherein hepatic fat accumulation is determined based on the level of hepatic triglyceride.

Embodiment 21. The method of embodiment 19 or 20, wherein hepatic fat accumulation is determined by quantifying stained liver images.

Embodiment 22. The method of embodiment 20 or 21, wherein the concentration of hepatic triglyceride in a treated tissue is less than 60 mg/g, less than 55 mg/g, less than 50 mg/g, less than 45 mg/g, less than 40 mg/g, less than 35 mg/g, less than 30 mg/g, less than 25 mg/g, or less than 20 mg/g.

Embodiment 23. The method of any of the preceding embodiments, further comprising preventing insulin resistance.

Embodiment 24. The method of embodiment 23, wherein insulin resistance is determined via an injected insulin tolerance test.

Embodiment 25. A method of ameliorating insulin resistance in a subject identified as in need thereof, comprising administering an effective amount of a compound of the formula:

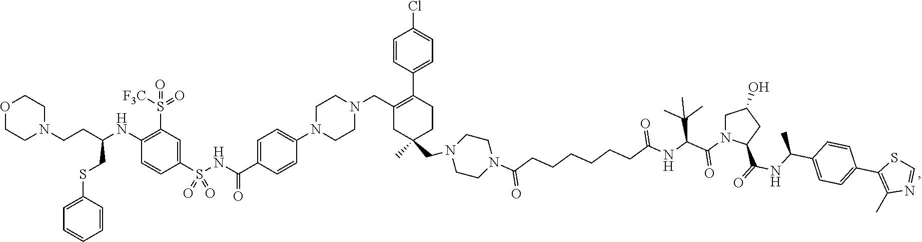 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt thereof, or a pharmaceutical composition thereof.

Embodiment 26. A method of ameliorating pre-diabetes, diabetes, and/or obesity in a subject identified as in need thereof, comprising administering an effective amount of a compound of the formula:

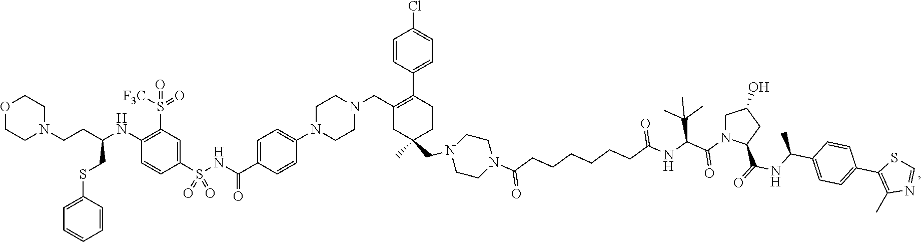 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt thereof, or a pharmaceutical composition thereof.

Embodiment 27. The method of any of embodiments 23-26, wherein the subject has a blood glucose level of less than 200 mg/dL, less than 190 mg/dL, less than 180 mg/dL, less than 170 mg/dL, less than 160 mg/dL, less than 150 mg/dL, less than 140 mg/dL, less than 130 mg/dL, less than 120 mg/dL, less than 90 mg/dL, less than 80 mg/dL, less than 70 mg/dL, less than 60 mg/dL, or less than 50 mg/dL 90 minutes after administration of insulin.

Embodiment 28. The method of any of embodiments 23-27, wherein the subject has a blood glucose level of about 5 mg/dL to about 100 mg/dL, about 5 mg/dL to about 90 mg/dL, about 5 mg/dL to about 80 mg/dL, about 5 mg/dL to about 70 mg/dL, about 5 mg/dL to about 60 mg/dL, or about 5 mg/d to about 50 mg/dL 90 minutes after administration of insulin.

Embodiment 29. A method of degrading B-cell lymphoma-2 (Bcl-2) and/or B-cell lymphoma-extra large (Bcl-xl) proteins, the method comprising contacting Bcl-2 and/or Bcl-xl with an effective amount of a compound of the formula:

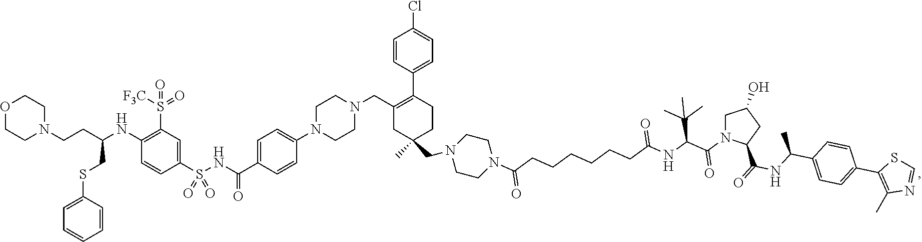 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt thereof, or a pharmaceutical composition thereof.

Embodiment 30. The method of embodiment 29, wherein the contacting is in vitro.

Embodiment 31. The method of embodiment 29 or 30, wherein the contacting is in vivo.

Embodiment 32. The method of any of embodiments 29-31, wherein the degrading of Bcl-2 and/or Bcl-xl is achieved in senescent cells.

Embodiment 33. The method of embodiment 32, wherein the senescent cells are liver cells or spleen cells.

Embodiment 34. The method of any of embodiments 29-33, wherein the protein is Bcl-xl.

Embodiment 35. The method of any of embodiments 29-33, wherein the protein is Bcl-2.

Embodiment 36. The method of any of embodiments 29-35, further comprising administering the compound to a subject.

Embodiment 37. A method of killing a senescent cell, the method comprising contacting the senescent cell with an effective amount of a compound of the formula:

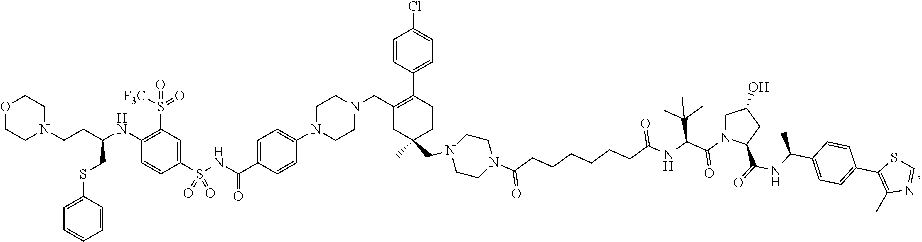 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]

or a pharmaceutically acceptable salt thereof, or a pharmaceutical composition thereof.

Embodiment 38. The method of embodiment 37, wherein the contacting is in vitro.

Embodiment 39. The method of embodiment 37, wherein the contacting is in vivo.

Embodiment 40. The method of any of embodiments 37-39, wherein the senescent cells are liver cells or spleen cells.

Embodiment 41. The method of any of embodiments 37-40, wherein the senescent cell is an ionizing radiation-induced senescent cell.

Embodiment 42. The method of embodiment 41, wherein the senescent cell is an ionizing radiation-induced senescent WI-38 fibroblast cell, ionizing radiation induced senescent human umbilical vein endothelial cell, or ionizing radiation-induced replicative senescent renal epithelial cell.

Embodiment 43. The method of any of embodiments 37-42, further comprising degrading Bcl-2 and/or Bcl-xl proteins.

Embodiment 44. The method of embodiment 43, wherein the protein is Bcl-xl.

Embodiment 45. The method of embodiment 43, wherein the protein is Bcl-2.

Embodiment 46. The method of any one of embodiments 37-45, wherein the compound reduces the viability of senescent cells by at least 5%, at least 10%, at least 15%, at least 20%, at least 25%, at least 30%, at least 40%, or at least 50% at a concentration of 100 nM or less.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method of treating a disease or disorder in a subject identified in need thereof, the method comprising administering an effective amount of a compound of the formula: 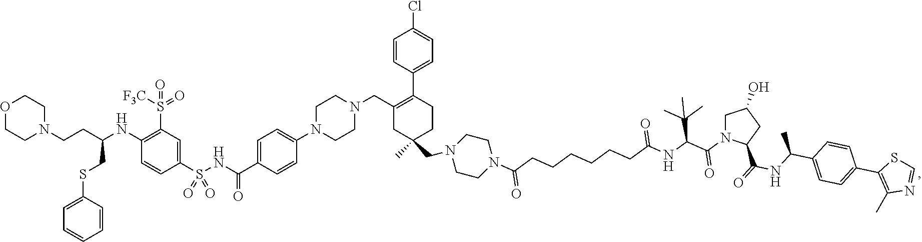 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]
or a pharmaceutically acceptable salt, solvate, hydrate, polymorph, co-crystal, tautomer, stereoisomer, isotopically labeled compound, or prodrug thereof, or a pharmaceutical composition thereof.

2. A method of preventing a disease or disorder in a subject identified in need thereof, the method comprising administering an effective amount of a compound of the formula: 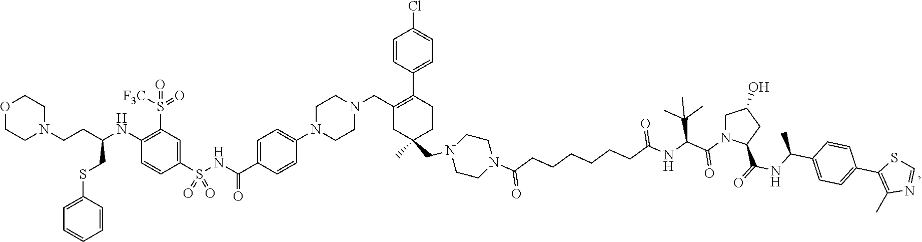 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]
or a pharmaceutically acceptable salt, solvate, hydrate, polymorph, co-crystal, tautomer, stereoisomer, isotopically labeled compound, or prodrug thereof, or a pharmaceutical composition thereof.

3. The method of claim 1 or 2 comprising administering an effective amount of the compound, or a pharmaceutically acceptable salt thereof, or a pharmaceutical composition thereof.

4. A method of treating a subject suffering from or susceptible to a disease or disorder, the method comprising administering an effective amount of a compound of the formula: 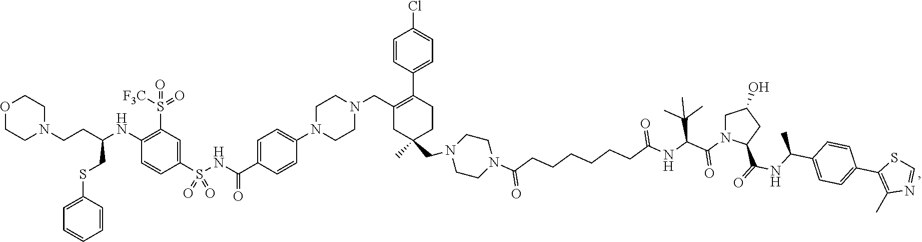 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]
or a pharmaceutically acceptable salt thereof, or a pharmaceutical composition thereof.

5. The method of any one of claims 1-4, wherein the disease is associated with senescent cell accumulation.

6. The method of any one of claims 1-5, wherein the disease is an age-related disease.

7. The method of any one of claims 1-6, wherein the disease is a liver disease.

8. The method of claim 7, wherein the disease is an alcohol-induced liver disease.

9. The method of claim 7, wherein the disease is a non-alcoholic fatty liver disease.

10. The method of claim 7, wherein the non-alcoholic fatty liver disease is a diabetes- and/or obesity-related non-alcoholic fatty liver disease.

11. The method of claim 7, wherein the disease is liver steatosis, non-alcoholic steatohepatitis (NASH), liver fibrosis, or cirrhosis.

12. The method of claim 11, wherein the disease is NASH, liver fibrosis, or cirrhosis.

13. The method of claim 7, wherein the disease is liver cancer.

14. The method of claim 13, wherein the liver cancer is hepatocellular carcinoma (HCC).

15. The method of any one of claims 1-12, wherein the disease is not cancer.

16. The method of claim 15, wherein the cancer is a solid tumor.

17. The method of any one of claims 1-16, wherein the subject is a mammal.

18. The method of claim 17, wherein the subject is a human.

19. The method of any one of claims 1-12 or 15-18, wherein the subject is not suffering from cancer.

20. The method of claim 19, wherein the cancer is a solid tumor.

21. The method of any one of claims 1-20, further comprising suppressing hepatic fat accumulation.

22. The method of claim 21, wherein hepatic fat accumulation is determined based on the level of hepatic triglyceride.

23. The method of claim 21 or 22, wherein hepatic fat accumulation is determined by quantifying stained liver images.

24. The method of claim 22 or 23, wherein the concentration of hepatic triglyceride in a treated tissue is less than 60 mg/g, less than 55 mg/g, less than 50 mg/g, less than 45 mg/g, less than 40 mg/g, less than 35 mg/g, less than 30 mg/g, less than 25 mg/g, or less than 20 mg/g.

25. The method of any one of claims 1-24, further comprising preventing insulin resistance.

26. The method of claim 25, wherein insulin resistance is determined via an injected insulin tolerance test.

27. A method of ameliorating insulin resistance in a subject identified as in need thereof, comprising administering an effective amount of a compound of the formula: 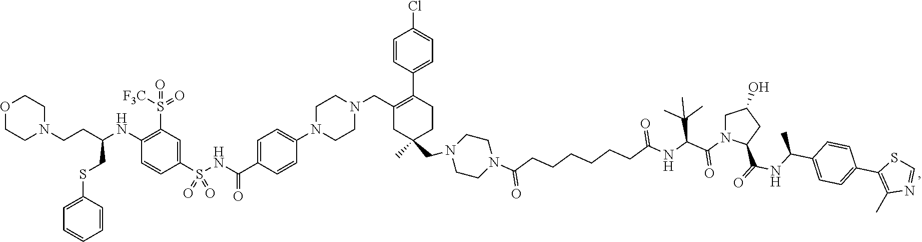 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]
or a pharmaceutically acceptable salt, solvate, hydrate, polymorph, co-crystal, tautomer, stereoisomer, isotopically labeled compound, or prodrug thereof, or a pharmaceutical composition thereof.

28. The method of claim 27 comprising administering an effective amount of the compound, or a pharmaceutically acceptable salt thereof, or a pharmaceutical composition thereof.

29. The method of claim 27 or 28, wherein the subject has a blood glucose level of less than 200 mg/dL, less than 190 mg/dL, less than 180 mg/dL, less than 170 mg/dL, less than 160 mg/dL, less than 150 mg/dL, less than 140 mg/dL, less than 130 mg/dL, less than 120 mg/dL, less than 90 mg/dL, less than 80 mg/dL, less than 70 mg/dL, less than 60 mg/dL, or less than 50 mg/dL 90 minutes after administration of insulin.

30. The method of claim 27 or 28, wherein the subject has a blood glucose level of about 5 mg/dL to about 100 mg/dL, about 5 mg/dL to about 90 mg/dL, about 5 mg/dL to about 80 mg/dL, about 5 mg/dL to about 70 mg/dL, about 5 mg/dL to about 60 mg/dL, or about 5 mg/d to about 50 mg/dL 90 minutes after administration of insulin.

31. A method of ameliorating pre-diabetes, diabetes, and/or obesity in a subject identified as in need thereof, comprising administering an effective amount of a compound of the formula: 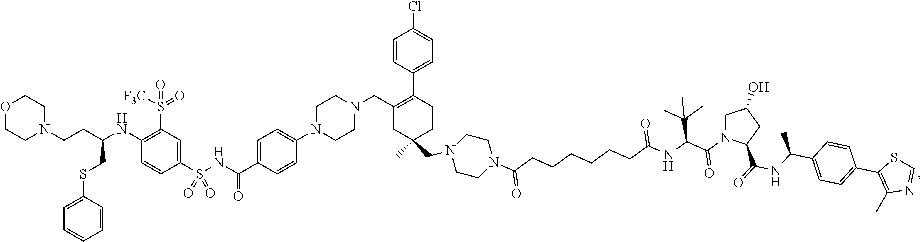 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]
or a pharmaceutically acceptable salt, solvate, hydrate, polymorph, co-crystal, tautomer, stereoisomer, isotopically labeled compound, or prodrug thereof, or a pharmaceutical composition thereof.

32. The method of claim 31 comprising administering an effective amount of the compound, or a pharmaceutically acceptable salt thereof, or a pharmaceutical composition thereof.

33. A method of degrading B-cell lymphoma-2 (Bcl-2) and/or B-cell lymphoma-extra large (Bcl-xl) proteins, the method comprising contacting Bcl-2 and/or Bcl-xl with an effective amount of a compound of the formula: 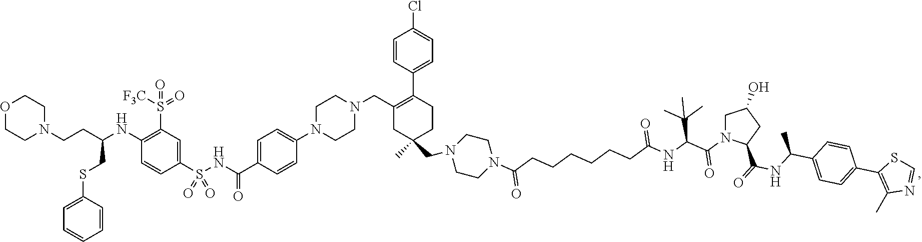 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]
or a pharmaceutically acceptable salt, solvate, hydrate, polymorph, co-crystal, tautomer, stereoisomer, isotopically labeled compound, or prodrug thereof, or a pharmaceutical composition thereof.

34. The method of claim 33 comprising contacting Bcl-2 and/or Bcl-xl with an effective amount of the compound, or a pharmaceutically acceptable salt thereof, or a pharmaceutical composition thereof.

35. The method of claim 33 or 34, wherein the contacting is in vitro.

36. The method of claim 33 or 34, wherein the contacting is in vivo.

37. The method of any one of claims 33-36, wherein the degrading of Bcl-2 and/or Bcl-xl is achieved in senescent cells.

38. The method of claim 37, wherein the senescent cells are liver cells or spleen cells.

39. The method of any one of claims 33-38, wherein the protein is Bcl-xl.

40. The method of any one of claims 33-38, wherein the protein is Bcl-2.

41. The method of any one of claims 33-40, further comprising administering the compound or a pharmaceutically acceptable salt, solvate, hydrate, polymorph, co-crystal, tautomer, stereoisomer, isotopically labeled compound, or prodrug thereof, or a pharmaceutical composition thereof, to a subject.

42. A method of killing a senescent cell, the method comprising contacting the senescent cell with an effective amount of a compound of the formula: 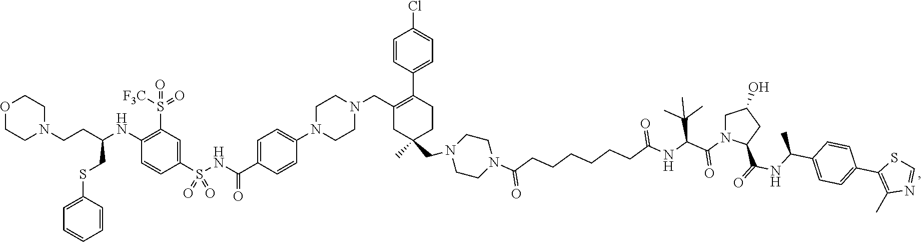 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]
or a pharmaceutically acceptable salt, solvate, hydrate, polymorph, co-crystal, tautomer, stereoisomer, isotopically labeled compound, or prodrug thereof, or a pharmaceutical composition thereof.

43. The method of claim 42 comprising contacting the senescent cell with an effective amount of the compound, or a pharmaceutically acceptable salt thereof, or a pharmaceutical composition thereof.

44. The method of claim 42 or 43, wherein the contacting is in vitro.

45. The method of claim 42 or 43, wherein the contacting is in vivo.

46. The method of any one of claims 42-45, wherein the senescent cells are liver cells or spleen cells.

47. The method of any one of claims 42-46, wherein the senescent cell is an ionizing radiation-induced senescent cell.

48. The method of claim 47, wherein the senescent cell is an ionizing radiation-induced senescent WI-38 fibroblast cell, ionizing radiation induced senescent human umbilical vein endothelial cell, or ionizing radiation-induced replicative senescent renal epithelial cell.

49. The method of any one of claims 42-48, further comprising degrading Bcl-2 and/or Bcl-xl proteins.

50. The method of claim 49, wherein the protein is Bcl-xl.

51. The method of claim 49, wherein the protein is Bcl-2.

52. The method of any one of claims 42-51, wherein the compound, or a pharmaceutically acceptable salt, solvate, hydrate, polymorph, co-crystal, tautomer, stereoisomer, isotopically labeled compound, or prodrug thereof, or a pharmaceutical composition thereof reduces the viability of senescent cells by at least 5%, at least 10%, at least 15%, at least 20%, at least 25%, at least 30%, at least 40%, or at least 50% at a concentration of 100 nM or less.

53. A kit comprising:
a first container comprising a compound of the formula: 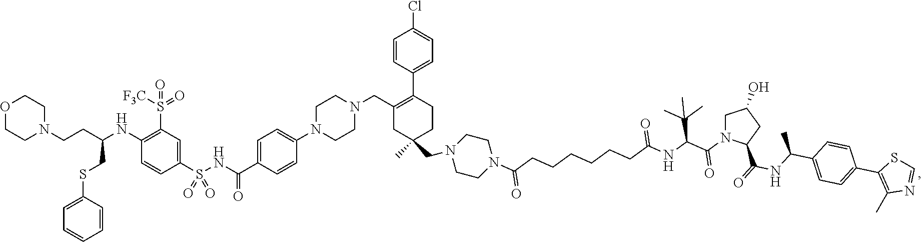 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1]
or a pharmaceutically acceptable salt, solvate, hydrate, polymorph, co-crystal, tautomer, stereoisomer, isotopically labeled compound, or prodrug thereof, or a pharmaceutical composition thereof; and
instructions for using the compound, or a pharmaceutically acceptable salt, solvate, hydrate, polymorph, co-crystal, tautomer, stereoisomer, isotopically labeled compound, or prodrug thereof, or a pharmaceutical composition thereof, in a method of any one of claims 1-52.
